(19) United States
(12) Patent Application Publication
(10) Pub. No.: US 2020/0087730 A1
(43) Pub. Date: Mar. 19, 2020

(54) METHODS OF IDENTIFYING AND TREATING TUMORS WITH SIGMA1 INHIBITORS

(71) Applicants: DREXEL UNIVERSITY, Philadelphia, PA (US); CONTEXT THERAPEUTICS, Philadelphia, PA (US)

(72) Inventors: Felix KIM, Philadelphia, PA (US); Halley OYER, Yardley, PA (US); John VAILLANCOURT, Montreal (CA)

(21) Appl. No.: 16/469,820

(22) PCT Filed: Dec. 14, 2017
(86) PCT No.: PCT/US2017/066265

§ 371 Date: Jun. 14, 2019

Related U.S. Application Data
(60) Provisional application No. 62/435,370, filed on Dec. 16, 2016.

(51) Int. Cl. C12Q 1/6886; A61P 35/00; A61K 31/155; G16H 50/20; G16B 40/10; A61K 31/4745

(52) U.S. Cl. CPC C12Q 1/6886; A61P 35/00; A61K 31/155; A61K 45/06; G16B 40/10; A61K 31/4745; G16H 50/20

(57) ABSTRACT

Methods and uses of using Sigma1 inhibitors are provide herein, including diagnostic methods for predicting or identifying quantitatively whether a human tumor is responsive or non-responsive to treatment with Sigma1 inhibition are also provided.

### CROSS-REFERENCE TO RELATED APPLICATIONS

This application claims priority under 35 U.S.C. § 119(e) to U.S. Provisional Application No. 62/435,370, filed Dec. 16, 2016, which is hereby incorporated by reference in its entirety.

### BACKGROUND

In 2012 there were 14 million new cases of cancer. Simultaneously, 8.2 million patients died from cancer. In addition to its effect on human lives, the treatment of this disease is projected to cost the U.S. about 207 billion dollars annually by the year 2020, if treatments continue to become more expensive and if cost saving methods are not found. Cancer destroys the lives and livelihoods of those affected by the disease. Therefore, methods to address both of these problems are in dire need.

One method by which these problems can be addressed is by better selecting treatments for individual patients. This can be done by using genetic analyses to determine preemptively which treatments are likely to be successful in a patient, and which treatments are likely to be ineffective in a patient. In so doing, it is possible to direct all resources towards those treatments that appear promising and away from those that are futile, with far greater precision and reliability. This will allow care providers to craft the best possible treatment plans for their patients, while simultaneously eliminating waste in the form of treatments previously shown to be ineffective for any given patient.

Sigma1, a protein encoded by the gene SIGMAR1, has been traditionally associated with pain relief, and its receptors were initially classified as opioids. However, more recent studies have shown this to be a mistaken characterization of the receptor. Furthermore, recent studies have shown that inhibition of this receptor holds immense promise in the treatment of certain cancers. Additionally, because of a long history of drugs in part inhibiting Sigma1 action (due to its association with pain relief), the potential side effects to inhibition are more thoroughly understood than many other treatment options, with an impressive safety record.

However, not all tumors are equally sensitive to compounds acting through Sigma1 inhibition. In fact, while many patients stand to benefit greatly from such an approach, many others patients are genetically predisposed to be resistant to such method. Thus, an approach designed to personalize treatment to each patient's own genetic profile is highly desirable. In order to facilitate the use of such compounds, and to avoid the waste that would come from their inappropriate use in resistant cases, the implementation of a methodology by which the differences can be analyzed and used to design and tailor individual treatment plans is necessary.

There is thus a need in the art for a set of genetic biomarkers that can be used or employed to identify a patient's individual and particular sensitivities to treatments operating through Sigma1 inhibition prior to, during, or after the use of such a compound. The present embodiments addresses this needs as well as others.

### BRIEF SUMMARY OF THE INVENTION

In certain embodiments, the methods for treating cancer in a subject using a Sigma1 inhibitor are provided. In other embodiments, methods for treating a subject with cancer by administering a Sigma1 inhibitor are provided. In yet other embodiments, the method comprises measuring the expression levels of at least three genes from a cancer tissue sample that is taken from a subject and treated/contacted with a Sigma1 inhibitor, wherein the at least three genes are selected from a group consisting of CDH1, CREB3L4, PIK3C2B, RNH43, SREBF1, and ZMYM2. In yet other embodiments, the method comprises calculating a signature score based upon the relative expression levels of the at least three genes, wherein increases in the relative expression levels of the at least three genes selected from a group consisting of CDH1, CREB3L4, PIK3C2B, RNH43, SREBF1, and ZMYM2 as compared to one or more controls identifies the subject as a subject that can be treated for the cancer with a Sigma1 inhibitor. In yet other embodiments, the method comprises administering to the identified subject a therapeutically effective amount of a Sigma1 inhibitor.

In certain embodiments, methods of determining whether a cancer in a subject can be treated using a Sigma1 inhibitor are provided. The method comprises measuring the expression levels of at least three genes from a cancer tissue sample that is taken from the subject and contacted with a Sigma1 inhibitor, wherein the at least three genes are selected from a group consisting of CDH1, CREB3L4, PIK3C2B, RNH43, SREBF1, and ZMYM2. The method further comprises calculating a signature score based upon the relative expression levels of the at least three genes, wherein increases in the relative expression levels of the at least three genes as compared to controls identifies the subject as having a cancer that can be treated with a Sigma1 inhibitor.

In certain embodiments, methods of treating cancer in a subject using a Sigma1 inhibitor are provided. In other embodiments, the method comprises administering a therapeutically effective amount of a Sigma1 inhibitor to a subject which cancer is identified as a cancer that can be treated with a Sigma1 inhibitor. In yet other embodiment, identifying the cancer as a cancer that can be treated with a Sigma1 inhibitor comprises measuring the expression levels of at least three genes from a cancer tissue sample that is taken from the subject and contacted with a Sigma1 inhibitor, wherein the at least three genes are selected from a group consisting of CDH1, CREB3L4, PIK3C2B, RNH43, SREBF1, and ZMYM2. In yet other embodiment, identifying the cancer as a cancer that can be treated with a Sigma1 inhibitor comprises calculating a signature score based upon the relative expression levels of the at least three genes, wherein increases in the relative expression levels of the at least three genes as compared to controls identifies the cancer as a cancer that can be treated with a Sigma1 inhibitor.

In certain embodiments, methods of identifying a tumor as being responsive to treatment with a Sigma1 inhibitor are provided. In other embodiments, the method comprises measuring the expression levels of at least three genes from a cancer tissue sample that is taken from the subject and treated/contacted with a Sigma1 inhibitor, wherein the at least three genes are selected from a group consisting of CDH1, CREB3L4, PIK3C2B, RNH43, SREBF1, and ZMYM2. In yet other embodiments, the method comprises calculating a signature score based upon the relative expression levels of the at least three genes, wherein increases in the relative expression levels of at least three of CDH1, CREB3L4, PIK3C2B, RNH43, SREBF1, and ZMYM2 compared to one or more controls identifies a tumor as being responsive to a Sigma1 inhibitor.

In certain embodiments, compositions comprising a plurality of polynucleotides specific for at least three of CDH1, CREB3L4, PIK3C2B, RNH43, SREBF1, and ZMYM2, wherein each polynucleotide independently optionally comprises a detectable fluorescent label or a non-naturally occurring nucleotide base are provided.

In certain embodiments, methods of measuring the expression levels of at least three genes from a cancer tissue sample that is taken from the subject and contacted with a Sigma1 inhibitor, wherein the at least three genes are selected from a group consisting of CDH1, CREB3L4, PIK3C2B, RNH43, SREBF1, and ZMYM2, wherein the sample is contacted with a composition comprising a plurality of polynucleotides specific for at least three of CDH1, CREB3L4, PIK3C2B, RNH43, SREBF1, and ZMYM2, under conditions sufficient to measure the expression of at least three of the genes. In other embodiments, the polynucleotides comprise a detectable label or a non-naturally occurring nucleotide are provided.

In certain embodiments, compositions comprising a group of double stranded polynucleotides, wherein the group comprises polynucleotides that are 20-100 nucleotides in length and can bind to the sequence of 3, 4, 5, or each of CDH1, CREB3L4, PIK3C2B, RNH43, SREBF1, and ZMYM2, wherein the double stranded polynucleotides comprise a detectable label or a non-naturally occurring nucleotide, or wherein the double stranded polynucleotides comprise a nucleic acid binding dye bound thereto are provided.

### BRIEF DESCRIPTION OF FIGURES

For the purpose of illustrating the invention, there are depicted in the drawings certain embodiments of the invention. However, the invention is not limited to the precise arrangements and instrumentalities of the embodiments depicted in the drawings.

FIG. 1 illustrates a non-limiting Sigma1 Signature.

FIG. 2 illustrates the five most sensitive and five most resistant NCI-60 cell lines to Sigma1 inhibition.

FIG. 3 illustrates presence of the Sigma1 Signature in patient samples and potential clinical utility. The table illustrates primary cancer sites with the 3-gene core signature expression up or down.

FIG. 4 illustrates a pre-clinical design, which establishes that CT-110 response signature associates with response to Sigma1 inhibition in vivo. Xenografts predicted to be sensitive (BT474) or resistant (MDAMB231) to Sigma1 inhibitor therapy were randomly assigned as (i) control or (ii) CT-110 as a Sigma1 inhibitor.

FIG. 5 illustrates a non-limiting clinical design, with patient treatment flow chart/decision tree.

### DETAILED DESCRIPTION

Embodiments described herein provide methods of identifying subjects that are suspected of having cancer or diagnosed with cancer that can be, or will be, treated with at least one Sigma1 inhibitor. Embodiments described herein also provide methods of identifying tumors or cancers that are responsive to Sigma1 inhibitors.

The embodiments described herein are based, in part, on the identification of a set of genes that: (a) are related to Sigma1 response in mammalian cells; (b) display coherence in their expression level in humans; and (c) whose expression levels collectively indicate whether a human tumor is likely to be responsive (sensitive) or non-responsive (resistant) to treatment with Sigma1 inhibitors. Accordingly, the embodiments provide methods for predicting quantitatively whether a human tumor will be responsive or non-responsive to treatment with Sigma1 inhibitors. In certain embodiments, the methods comprise (a) measuring, in a tissue sample from a human tumor, the relative expression level of at least two genes in a Sigma1 signature, wherein the Sigma1 signature consists of the following seven genes (denoted by the Human Gene Organization (HUGO) gene symbol): CDH1, CREB3L4, PIK3C2B, RNF43, SREBF1, ZMYM2; and (b) calculating a Sigma1 signature score which is determined accordingly:

| Gene | Change in expression determining sensitivity |
|---|---|
| CHD1 | increase |
| CREB3L4 | increase |
| PIK3C2B | increase |
| RNF43 | increase |
| SREBF1 | increase |
| ZMYM2 | increase |

In certain embodiments, the expression of a combination of at least three, four, five, or six of these genes are determined. In certain embodiments, the expression of the combination of all genes CDH1, CREB3L4, PIK3C2B, RNF43, SREBF1 and ZMYM2 is determined. In certain embodiments, when 3, 4, 5, or all of the genes are increased as compared to a control, the subject or tumor is identified as being sensitive to a Sigma1 inhibitor. The control can be a normal benign control and can be from the same tissue type and/or from the same subject. In certain embodiments, the control is not from the same subject, but is from a pool sample set that is considered benign or normal. In certain embodiments, the control is a tumor sample set that has previously been found to be responsive to a Sigma1 inhibitor. The control sample can be a single sample or a pooled sample.

In certain embodiments, the expression of at least another one gene selected from the group comprising AR, BRCA2, EGFR, ERBB2, ERBB3, FASN and PTEN is determined. In certain embodiments, the change expression is determined according to the following table:

| Gene | Change in expression determining sensitivity |
|---|---|
| AR | increase |
| BRCA2 | increase |
| EGFR | increase |
| ERBB2 | increase |
| ERBB3 | increase |
| FASN | increase |
| PTEN | decrease |

In certain embodiments, the expression of a combination of at least 3, 4, 5 or 6 of these genes is determined. In certain embodiments, the expression of the combination of all genes comprising AR, EGFR, BRCA2, ERBB2, ERBB3, FASN and PTEN is determined, and the sample is said to be responsive when the expression levels are increased or decreased as indicated herein.

In certain embodiments, Sigma1 signature expression level above a defined threshold indicates that the tumor is likely to be responsive to a Sigma1 inhibitor, and a Sigma1 signature expression level below a defined threshold indicates that the tumor is likely to be non-responsive to a Sigma1 inhibitor. In certain embodiments, the collective increase is at least 10%, 20%, 30%, 40%, 50%, 60%, 70%, 80%, 90%, 100%, 200%, 300%, 400%, and the like. The term “Sigma1 signature expression level” takes into account the expression levels of all six genes in the Sigma1 signature, or a three-, four-, five-, or six-gene subset thereof. The Sigma1 signature score may be directly correlated or inversely correlated with the Sigma1 signature expression level, depending on the gene expression assay method and the units of measurement used. The increase expression can, for example, be a sum of the changes in expression.

In certain embodiments, the gene signature of 3, 4, 5, or each of CDH1, CREB3L4, PIK3C2B, RNF43, SREBF1 and ZMYM2 is used in conjunction with a gene signature of AR, EGFR, BRCA2, ERBB2, ERBB3, FASN or PTEN. The changes in expression that are indicative of being responsive to Sigma1 inhibition are described herein and above. In certain embodiments, an increase expression (collective or individually) of 3, 4, 5, or each of CDH1, CREB3L4, PIK3C2B, RNF43, SREBF1 and ZMYM2 and a decrease in expression PTEN indicates that the tumor will be responsive to Sigma1 inhibition.

In certain embodiments, an increase expression (collective or individually) of 3, 4, 5, or each of CDH1, CREB3L4, PIK3C2B, RNF43, SREBF1 and ZMYM2 and an increase in expression of (collectively or individually) 1, 2, 3, 4, 5, or each of AR, EGFR, BRCA2, ERBB2, ERBB3, and FASN indicates that the tumor will be responsive to Sigma1 inhibition.

In certain embodiments, an increase expression (collective or individually) of 3, 4, 5, or each of CDH1, CREB3L4, PIK3C2B, RNF43, SREBF1 and ZMYM2, an increase in expression of (collectively or individually) 1, 3, 4, or each of AR, EGFR, BRCA2, ERBB2, ERBB3, and FASN and a decreased in expression of PTEN indicates that the tumor will be responsive to Sigma1 inhibition.

In certain embodiments, the method includes normalizing the relative expression level of each gene in the Sigma1 signature using an internal gene expression standard for each sample. In certain embodiments, the method includes performing a threshold determination analysis, thereby generating a defined threshold. The threshold determination analysis can include a receiver operator characteristic curve analysis. The relative gene expression level for each gene in the Sigma1 signature can be obtained by measuring the messenger RNA (mRNA) level for that gene. Suitable methods for measuring mRNA levels in tumor tissue samples include DNA microarray analysis, quantitative reverse transcriptase polymerase chain reaction (qRT-PCR), e.g., TAQMAN® assays, quantitative nuclease protection assays, and nuclear “barcode” assays, e.g., the NANOSTRING® nCOUNTER™ assay.

In certain embodiments, the invention provides a probe set (e.g., a PCR primer set) that comprises probes (e.g., PCR primer pairs) for measuring expression of each gene in the Sigma1 signature gene set. The probe set can be incorporated into a diagnostic test kit are provided.

In certain embodiments, methods of treating a cancer patient are provided. The method comprises: (a) determining whether the patient is likely to be responsive to Sigma1 inhibition by: (1) measuring, in a tissue sample from a tumor in the patient, the relative expression level of at least six genes in a Sigma1 signature consisting of the following genes CDH1, CREB3L4, PIK3C2B, RNF43, SREBF1, ZMYM2; and (b) calculating a Sigma1 signature score is determined:

### Table 1

| Gene | Change in expression determining sensitivity |
|---|---|
| CHD1 | increase |
| CREB3L4 | increase |
| PIK3C2B | increase |
| RNF43 | increase |
| SREBF1 | increase |
| ZMYM2 | increase |

In certain embodiments, the expression of a combination of at least 3, 4, 5 or 6 of these genes is determined. In certain embodiments, (a) the expression of the combination of all genes CDH11, CREB3L4, PIK3C2B. RNF43, SREBF1 and ZMYM2 is determined; a Sigma1 signature expression level above a defined threshold indicates that the tumor is likely to be responsive to Sigma1 inhibition, and a Sigma1 signature expression level below the defined threshold indicates that the tumor is likely to be resistant to Sigma1 inhibition; and (b) administering to the patient a therapeutically effective amount of Sigma1 inhibition if step (a) yields a result indicating that the tumor is likely to be responsive to Sigma1 inhibition. In certain embodiments, an increase in the expression of each gene in Table 1 indicates that the tumor will not be resistant to, or will be susceptible to, Sigma1 inhibition. In certain embodiments, an increase in expression of 3, 4, 5 or each of CDH1, CREB3L4, PIK3C2B, RNF43, SREBF1 and ZMYM2 indicates that the tumor will respond to Sigma1 inhibition treatment. In certain embodiments, the increase in expression is compared to the gene expression of a tumor sample from the subject prior to treatment with the Sigma1 inhibitor. In certain embodiments, the expression levels are compared to a control sample that is known to respond to Sigma1 inhibition and an increase in expression that is within about 10%, 2090, or 309% of the control indicates that the tumor will respond to the Sigma1 inhibitor and that the patient should be treated, or should be continued to be treated with the Sigma1 inhibitor.

In certain embodiments, Sigma1 signature expression level for the genes in any of the Tables described herein above a defined threshold indicates that the tumor is likely to be responsive to a Sigma1 inhibitor, and a Sigma1 signature expression level below a defined threshold indicates that the tumor is likely to be non-responsive to a Sigma1 inhibitor. In certain embodiments, the increase is at least 10%, 20%, 30%, 40%, 50%, 60%, 70%, 80%, 90%, 100%, 200%, 300%, 400%, and the like of 3, 4, 5, or each of CDH1, CREB3L4, PIK3C2B, RNF43, SREBF1 and ZMYM2.

Exemplary human tumors and cancers which responsiveness to treatment and/or that can be treated using the approaches disclosed herein include bladder, breast, central nervous, cervical, colon, esophageal, head and neck, hematopoietic, intestinal, lung, ocular, oral, ovarian, pancreatic, prostate, rectal, renal, skin, soft tissue, stomach, thyroid, urinary tract, and uterine cancer.

The expression levels of the genes of the Sigma1 signature can be used collectively as a multi-gene predictive biomarker for classifying human tumors according to their likelihood of responding to treatment with Sigma1 inhibitors. Such classification of tumors is useful for identifying human patients who are suitable candidates for treatment with Sigma1 inhibitors in a clinical setting.

Also provided herein are compositions comprising a plurality of polynucleotides, wherein the composition comprises polynucleotides specific for at least three of CDH1, CREB3L4, PIK3C2B, RNH43, SREBF1, and ZMYM2. In certain embodiments, the polynucleotide comprises a detectable label. The detectable label can be, for example, a fluorescent label. In certain embodiments, the polynucleotides comprises at least one non-naturally occurring nucleotide base. In certain embodiments, the non-naturally occurring nucleotide base is a locked nucleic acid, which can also be referred to as “LNA”. Examples of non-naturally occurring or labeled nucleotides can be found, for example, in U.S. Pat. Nos. 6,303,315, 6,670,461, 6,794,499, 7,034,133, 7,572,582, 8,080,644, 8,153,365, 8,293,684, 9,464,106, each of which is incorporated by reference in its entirety. In certain embodiments, the polynucleotide is a primer that can amplify the gene that that the polynucleotide is specific for. The composition can have a plurality of polynucleotides that are specific for different target sequences. Thus, in certain embodiments, the composition comprises polynucleotides, wherein the polynucleotides are specific for 1, 2, 3, 4, or 5 of CDH1, CREB3L4, PIK3C2B, RNH43, SREBF1, or ZMYM2. In certain embodiments, the composition comprises polynucleotides that are specific for each of the target gene sequences.

In certain embodiments, the composition further comprises a polynucleotide specific for at least 1, 2, 3, 4, 5, or 6 of AR, BRCA2, EGFR, ERBB2, ERBB3, FASN, and PTEN. In certain embodiments, the composition further comprises a polynucleotide specific for AR, BRCA2, EGFR, ERBB2, ERBB3, FASN, and PTEN.

The polynucleotides can be of varying length. For example, the length of a polynucleotide specific for CDH1 can be different from the length of a polynucleotide specific for CREB3L4 or any of the other target sequences. That is, each polynucleotide that is specific for a target sequence can be a different length as compared to another polynucleotide that is specific for a different target sequence. In certain embodiments, the polynucleotide is, independently, about 15 to about 200, about 15 to about 150, about 15 to about 100, about 15 to about 75, about 15 to about 50, about 15 to about 45, about 15 to about 35, about 15 to about 30, about 20 to about 200, about 20 to about 150, about 20 to about 100, about 20 to about 75, about 20 to about 50 nucleotides in length, about 20 to about 45, about 20 to about 40, about 25 to about 200, about 25 to about 150, about 25 to about 100, about 25 to about 75, about 25 to about 50, about 25 to about 45, about 25 to about 40, about 30 to about 200, about 30 to about 150, about 30 to about 100, about 30 to about 75, about 30 to about 50, about 30 to about 40, about 35 to about 200, about 35 to about 150, about 35 to about 100, about 35 to about 75, about 35 to about 50, about 40 to about 200, about 40 to about 150, about 40 to about 100, about 40 to about 75, about 40 to about 50 nucleotides in length. As described herein, these nucleic acid molecules can be used as primers in an amplification method or probes to detect the presence and/or measure the expression of a target sequence in a qualitative assay.

In certain embodiments the composition comprises an amplification product that has incorporated the polynucleotides described herein. When acting as a primer or probe the polynucleotide can bind to the target sequence to form a hybrid or a double stranded sequence, if for example, after an amplification process is performed, such as PCR, RT-PCR, qPCR, or Loop-mediated isothermal amplification (LAMP). In certain embodiments, the polynucleotide sequence does not have a detectable label, but instead the amplification product is detected by adding a detectable reagent that can bind to or interact with the amplification product. Examples of such detectable reagents include, but are not limited to, SYBR green (N′,N′-dimethyl-N-[4-[(E)-(3-methyl-1,3-benzothiazol-2-ylidene)methyl]-1-phenylquinolin-1-ium-2-yl]-N-propylpropane-1,3-diamine), SYBR Green II, SYBR Green I, SYBR Gold, SYT09, EvaGreen, DIAMOND™ Nucleic Acid Dye (Promega), and the like. Methods of detecting amplification products with nucleic acid dyes, such as those described herein, can be performed according to various protocols.

In certain embodiments, the length of the amplification product is, independently, about 15 to about 200, about 15 to about 150, about 15 to about 100, about 15 to about 75, about 15 to about 50, about 15 to about 45, about 15 to about 35, about 15 to about 30, about 20 to about 200, about 20 to about 150, about 20 to about 100, about 20 to about 75, about 20 to about 50 nucleotides in length, about 20 to about 45, about 20 to about 40, about 25 to about 200, about 25 to about 150, about 25 to about 100, about 25 to about 75, about 25 to about 50, about 25 to about 45, about 25 to about 40, about 30 to about 200, about 30 to about 150, about 30 to about 100, about 30 to about 75, about 30 to about 50, about 30 to about 40, about 35 to about 200, about 35 to about 150, about 35 to about 100, about 35 to about 75, about 35 to about 50, about 40 to about 200, about 40 to about 150, about 40 to about 100, about 40 to about 75, about 40 to about 50 nucleotides in length. The amplification product can be a single strand or double stranded polynucleotide sequence. In certain embodiments, the amplification product comprises the non-naturally occurring nucleotide base and/or the detectable label. In certain embodiments, the amplification product comprises a nucleic acid binding dye bound to the amplification product as described herein.

Accordingly, in certain embodiments, methods of measuring the expression levels of at least three genes from a cancer tissue sample that is taken from the subject and contacted with a Sigma1 inhibitor are provided, wherein the at least three genes are selected from a group consisting of CDH1, CREB3L4, PIK3C2B, RNH43, SREBF1, and ZMYM2, wherein the sample is contacted with a composition comprising a plurality of polynucleotides specific for 3, 4, 5, or each of CDH1, CREB3L4, PIK3C2B, RNH43, SREBF1, and ZMYM2, under conditions sufficient to measure the expression of 3, 4, 5, or each of the genes. In certain embodiments, the sample is contacted with the different specific polynucleotides in different reactions. For example, the expression of CDH1 can be measured in one reaction and the expression of one or more of the other target sequences described herein can be measured in another reaction, or each is measured in its own reaction separately. In certain embodiments, different detectable labels can be used to measure the expression of multiple target sequences in the same reaction. The use of CDH1 in the example above is merely for illustrative purposes only and any of the other target genes described herein could be used.

In certain embodiments, the method further comprises measuring the expression of at least 1, 2, 3, 4, 5, 6, or each of AR, BRCA2, EGFR, ERBB2, ERBB3, FASN, and PTEN. In certain embodiments of the methods provided herein, the composition contacted with the sample comprises a plurality of polynucleotides specific for 1, 2, 3, 4, 5, 6 or each of AR, BRCA2, EGFR, ERBB2, ERBB3, FASN, and PTEN.

As described herein, the compositions used in the methods can comprise at least one non-naturally occurring nucleotide base, such as a LNA, or a detectable label. The amplification products can also be detected with nucleic acid binding dyes, including, but not limited to, those described herein.

Accordingly, in certain embodiments, compositions comprising a group of double stranded polynucleotides are provided, wherein the group comprises polynucleotides 20-100 nucleotides in length that comprise a nucleic acid molecule that can bind to the sequence of 3, 4, 5, or each of CDH1, CREB3L4, PIK3C2B, RNH43, SREBF1, and ZMYM2, wherein the double stranded polynucleotides comprise a detectable label or a non-naturally occurring nucleotide or the double stranded polynucleotides comprise a nucleic acid binding dye bound to the double stranded polynucleotides. In certain embodiments, the composition further comprises double stranded polynucleotides that comprise a nucleic acid molecule that can bind to 1, 2, 3, 5, 6, or each of AR, BRCA2, EGFR, ERBB2, ERBB3, FASN, and PTEN. In certain embodiments, the dye is SYBR Green or dye described herein. In certain embodiments, the double stranded polynucleotides are, independently, about 15 to about 200, about 15 to about 150, about 15 to about 100, about 15 to about 75, about 15 to about 50, about 15 to about 45, about 15 to about 35, about 15 to about 30, about 20 to about 200, about 20 to about 150, about 20 to about 100, about 20 to about 75, about 20 to about 50 nucleotides in length, about 20 to about 45, about 20 to about 40, about 25 to about 200, about 25 to about 150, about 25 to about 100, about 25 to about 75, about 25 to about 50, about 25 to about 45, about 25 to about 40, about 30 to about 200, about 30 to about 150, about 30 to about 100, about 30 to about 75, about 30 to about 50, about 30 to about 40, about 35 to about 200, about 35 to about 150, about 35 to about 100, about 35 to about 75, about 35 to about 50, about 40 to about 200, about 40 to about 150, about 40 to about 100, about 40 to about 75, about 40 to about 50 nucleotides (base pairs) in length. The amplification products for the different target sequences can be different lengths.

As used herein a nucleic acid molecule that can bind to a target sequence, such as a gene described herein, is a nucleic acid molecule that can bind under stringent conditions, such as those found using PCR, qPCR, and the like. Examples of such nucleic acid molecules are primers and probes.

Expression levels can also be quantified by other methods such as arrays and the like.

As used herein, Sigma1 means the sigma non-opioid intracellular receptor 1, also referred to as Sigma1 receptor, Sigma1R, Sigma1, SIGMAR1, opioid receptor sigma1 (OPRS1), ALS16, SRBP, SR-BP, SIG-1R, SR-BP1, sigma1R, hSigmaR1, Sigma1 protein, Sigma1 chaperone protein or Sigma1 scaffolding protein, any splice variant thereof, any isoform thereof, or any combination thereof.

As used herein, Sigma1 inhibitor means any small molecule, peptide, antibody, or combination thereof that binds to Sigma1 and/or modifies the activity or biological function of Sigma1.

As used herein, “CT-110”, a Sigma1 inhibitor, means 1-(3-(4-fluorophenoxy) propyl)-3-(4-chlorophenyl)guanidine, which has the following chemical structure, including salts and polymorphs thereof:

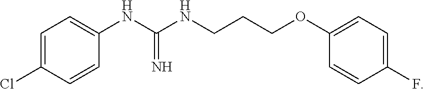 [SMILES: N=C(NCCCOc1ccc(F)cc1)Nc1ccc(Cl)cc1]

As used herein, “probe” means a molecule that can be used for measuring the expression of a particular gene. Exemplary probes include PCR primers gene-specific DNA oligonucleotide probes, such as microarray probes affixed to a microarray substrate; quantitative nuclease protection assay probes such as qNPA™ probes; probes linked to molecular barcodes, such as NanoString® nCounter™ probes; and probes affixed to beads.

The present application incorporates all teachings and disclosures of U.S. Patent Appl. Publ. No. 2015/0299804, which is incorporated by reference in its entirety herein.

In certain embodiments, the Sigma1 inhibitor is any one of the compounds described in U.S. Patent Appl. Publ. No. 2015/0166472, which is hereby incorporated by reference in its entirety.

In certain embodiments, the Sigma1 inhibitor is selected from the group consisting of CT-110, IPAG, haloperidol, PB28, rimcazole, and

(i) a compound of Formula (I)

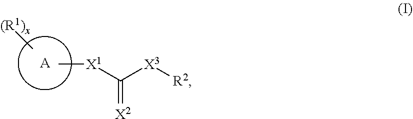

wherein in (I):
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇) heterocycloalkyl group, optionally substituted with 0-2 R¹ groups; each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X¹ is —CH₂—, —S—, —O— or —(NR²)—;
X² is ═CH₂, ═S, ═O or ═NR²; and
X³ is —S—, —O—, or —NR²—; and
(ii) a compound of Formula (II): R^{A}—R^{B} (II), wherein in (II):
R^{A} is selected from the group consisting of

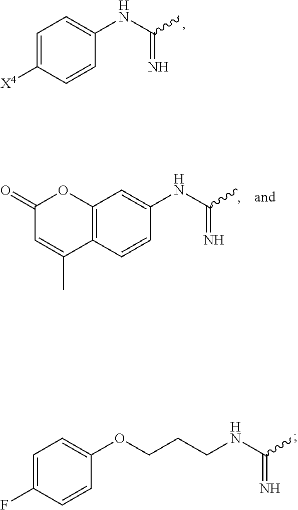

and
R^{B} is selected from the group consisting of:

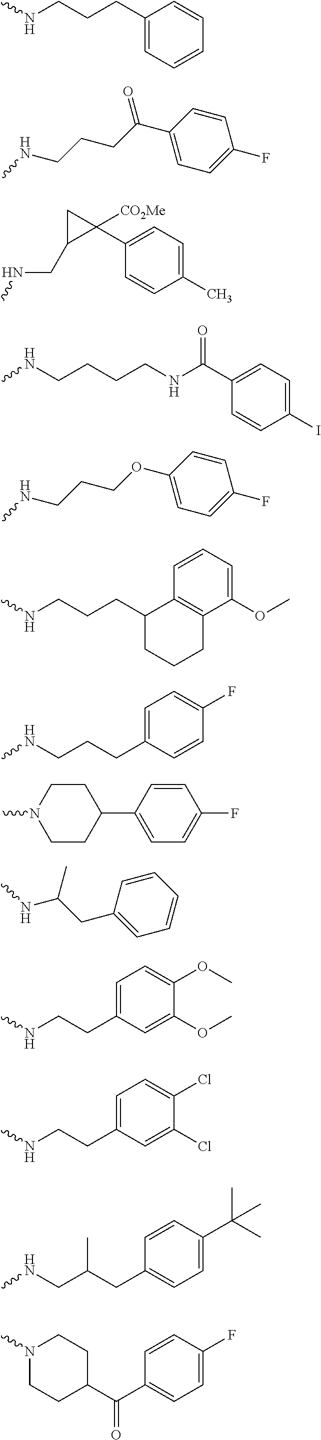

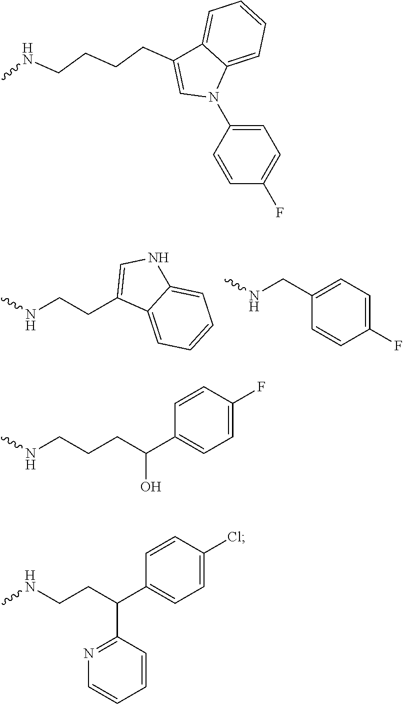

a pharmaceutically acceptable salt or solvate thereof, a N-oxide thereof, and any combinations thereof.

In certain embodiments, the compound is a compound of Formula (I-A), or a pharmaceutically acceptable salt or solvate thereof:

R^{A}—R^{B} (I-A),

wherein in (I-A):
R^{A} is selected from the group consisting of

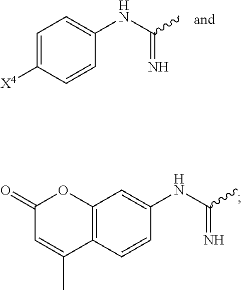

X⁴ is selected from the group consisting of OMe, F, Cl, Br, and I; and
R^{B} is selected from the group consisting of:

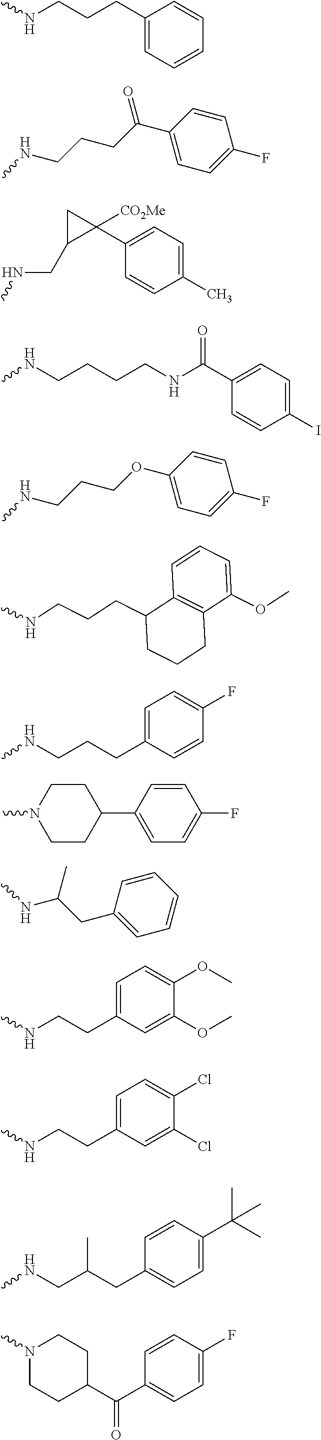

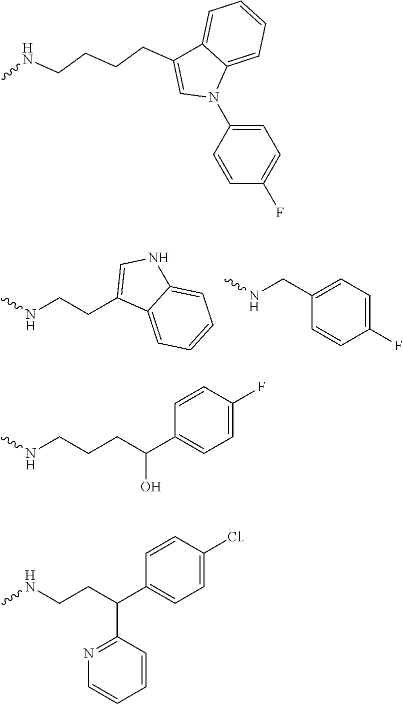

In certain embodiments, the compound is a compound of formula (I-A):

### R^{A} is

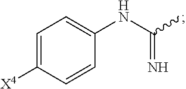

X⁴ is selected from the group consisting of F, Cl, Br, and I; and
R^{B} is selected from the group consisting of:

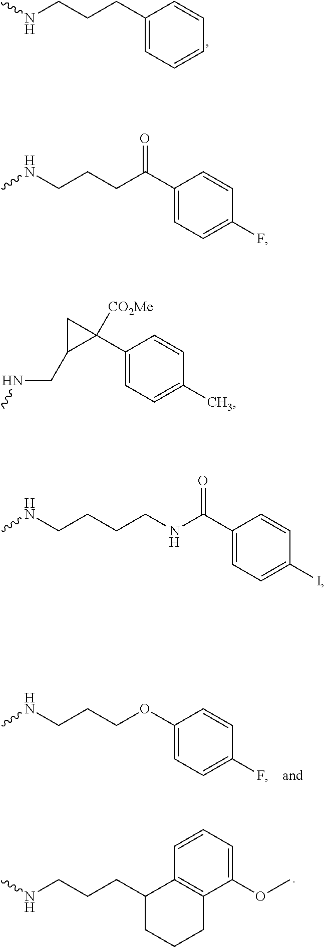

In certain embodiments, the compound is a compound of formula (I-A):

### R^{A} is

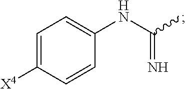

X⁴ is selected from the group consisting of F, Cl, Br, and I; and
R^{B} is selected from the group consisting of:

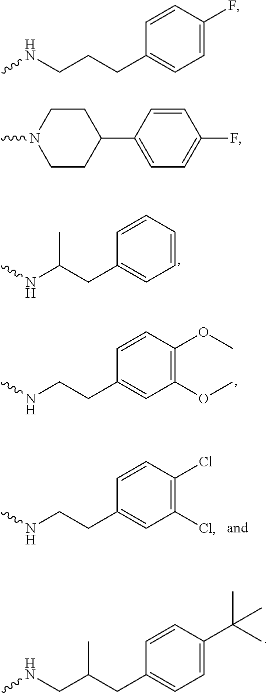

In certain embodiments, the compound is a compound of formula (I-A):

### R^{A} is

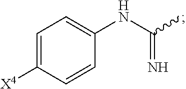

X⁴ is selected from the group consisting of F, Cl, Br, and I; and
R^{B} is selected from the group consisting of:

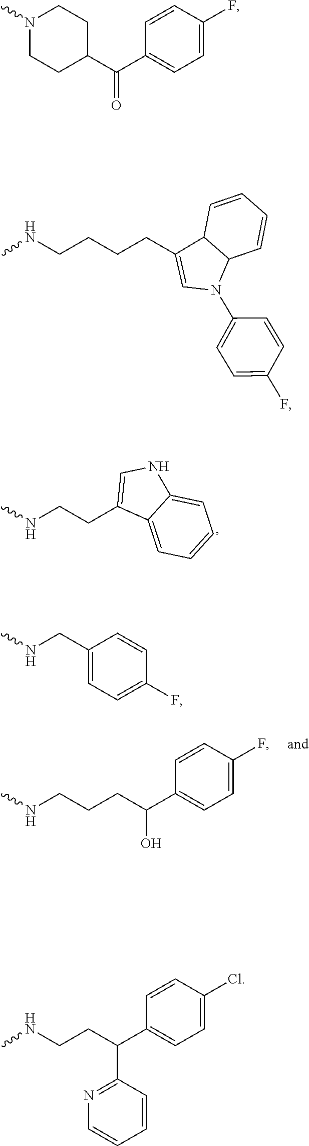

In certain embodiments, the compound is a compound of formula (II):

### R^{A} is

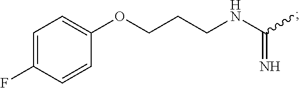

and
R^{B} is selected from the group consisting of:

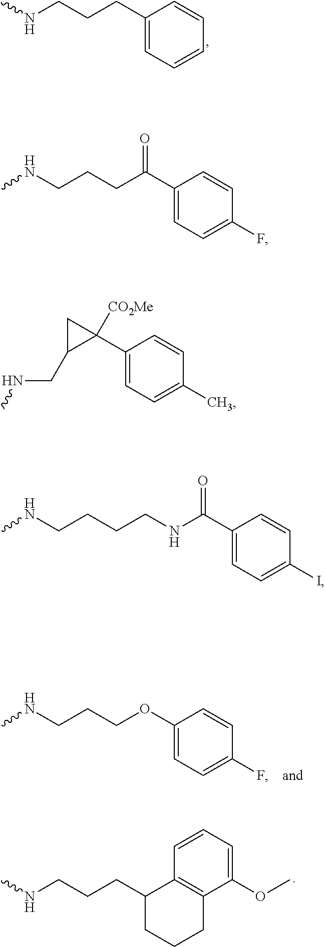

In certain embodiments, the compound is a compound of formula (II):

### R^{A} is

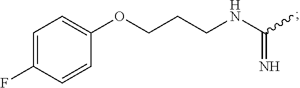

and
R^{B} is selected from the group consisting of:

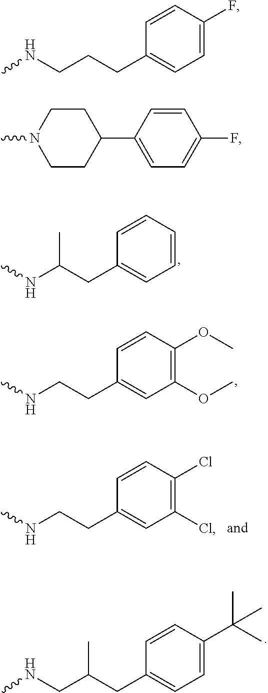

In certain embodiments, the compound is a compound of formula (II):

### R^{A} is

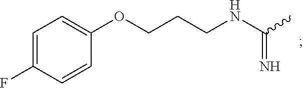

and
R^{B} is selected from the group consisting of:

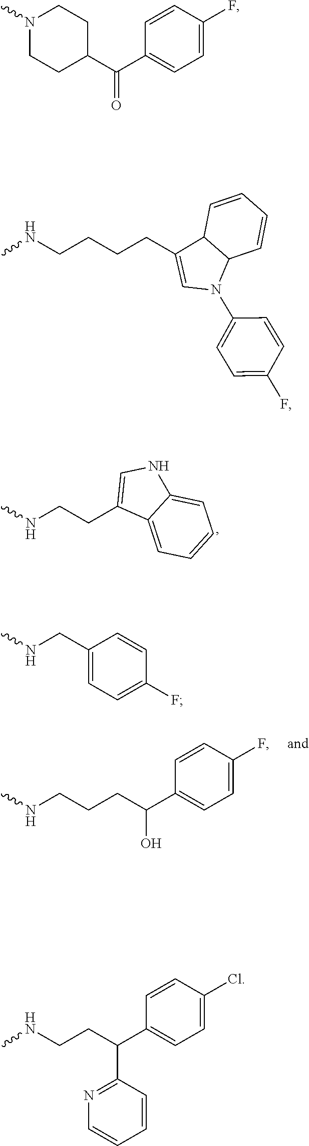

In certain embodiments, the compound is a compound of Formula (I-A):

### R^{A} is

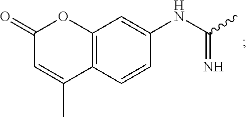

and
R^{B} is selected from the group consisting of:

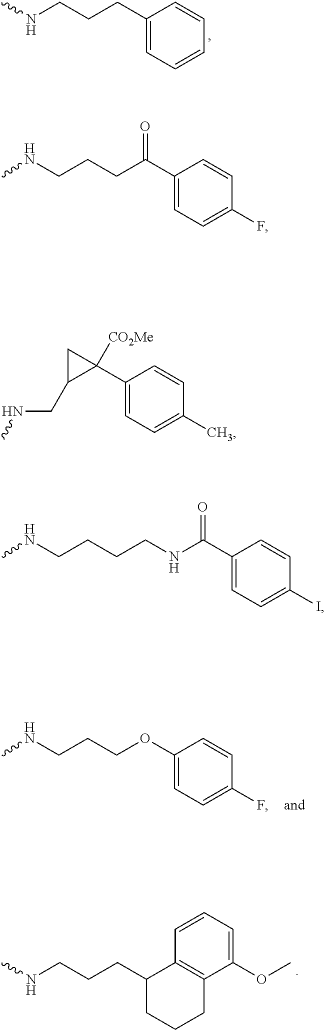

In certain embodiments, the compound is a compound of Formula (I-A):

### R^{A} is

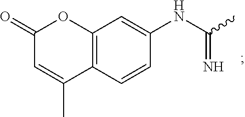

and
R^{B} is selected from the group consisting of:

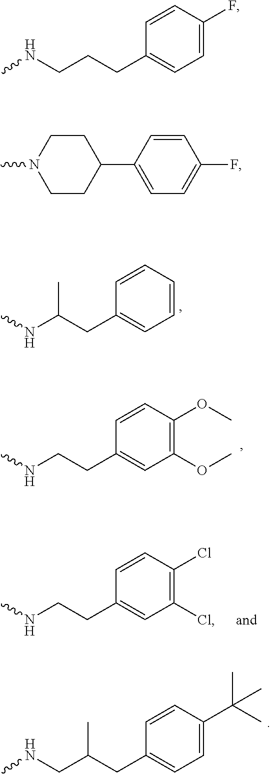

In certain embodiments, the compound is a compound of Formula (I-A):

### R^{A} is

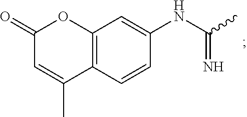

and
R^{B} is selected from the group consisting of:

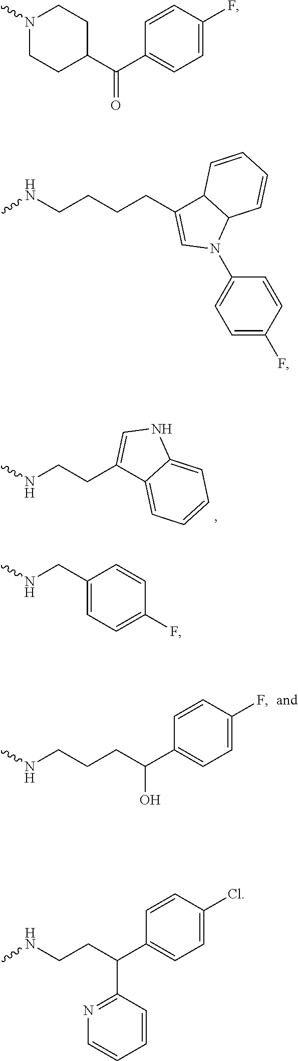

In certain embodiments, the compound is a compound is a compound of formula (I-B), or a pharmaceutically acceptable salt or solvate thereof:

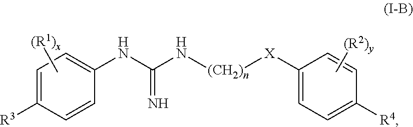

wherein in (I-B):
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵, —OC₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I; each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X is selected from the group consisting of CH₂, C═O, and O;
n is an integer from 1-3;
x is an integer from 0-4; and
y is an integer from 0-4.

In certain embodiments, the compound is selected from the group consisting of:
1-(3-(4-fluorophenoxy)propyl)-3-(4-iodophenyl)guanidine (Compound A),
1-(3-(4-fluorophenoxy)propyl)-3-(4-methoxyphenyl)guanidine (Compound B),
1-(n-propyl)-3-(4-iodophenyl)guanidine (Compound C),
1-(n-propyl)-3-(4-methoxyphenyl)guanidine (Compound D),
1,3-bis(3-(4-fluorophenoxy)propyl)guanidine (Compound E),
1-(3-(4-fluorophenoxy)propyl)-3-(4-trifluoromethylphenyl)guanidine (Compound F),
1-(3-(4-fluorophenoxy)propyl)-3-(4-chlorophenyl)guanidine (Compound G), and
1-(3-(4-fluorophenoxy)propyl)-3-(4-methyl-2-oxo-2Hchromen-7-yl)guanidine) (Compound H),
   or a pharmaceutically acceptable salt or solvate thereof, and any combinations thereof.

In certain embodiments, the compound is selected from the group consisting of CT-110 (1-(3-(4-fluorophenoxy)propyl)-3-(4-chlorophenyl)guanidine), IPAG (1-(4-Iodophenyl)-3-(2-adamantyl) guanidine), haloperidol (4-[4-(4-Chlorophenyl)-4-hydroxy-1-piperidinyl]-1-(4-fluorophenyl)-1-butanone hydrochloride), PB28 (1-Cyclohexyl-4-[3-(1,2,3,4-tetrahydro-5-methoxy-1-naphthalenyl)propyl] piperazine dihydrochloride), and rimcazole (9-[3-(cis-3,5-Dimethyl-1-piperazinyl)propyl]-9H-carbazole dihydrochloride).

In certain embodiments, the compound is any Sigma1 inhibitor recited in U.S. Patent Application Publication No. 2015/0166472, which is incorporated herein in its entirety by reference.

In certain embodiments, methods of treatment are provided wherein the subject or tumor sample is treated and/or contacted with a Sigma1 inhibitor as described herein. In certain embodiments, the compound is administered in a pharmaceutical composition. In certain embodiments, the compound is administered orally, topically, or intravenously, or by any other route described herein. For example, routes of administration of any of the compositions of the disclosure include oral, nasal, rectal, intravaginal, parenteral, buccal, sublingual, or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

The compounds, or pharmaceutically acceptable salts thereof, may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose. The unit dosage form may also comprise one or more additional therapeutics, such as, but not limited to, those described herein.

In certain embodiments, the subject is a mammal. In certain embodiments, the mammal is a human. In certain embodiments, the subject is a subject in need thereof.

In certain embodiments, the sample is a “cell sample,” “tissue sample,” or a composition comprising an isolated cell or plurality of cells. The sample may comprise an individual cell, a composition comprising a plurality of cells, a tissue sample taken from a subject suspected of having or diagnosed with cancer. The sample may be freshly obtained, formalin fixed, alcohol-fixed and/or paraffin embedded. The cell sample may be a biopsy isolated from a subject who has been diagnosed with, is suspected of having, or identified as having cancer. The sample may comprise a tissue from a brushing, scraping, punch biopsy, pinch biopsy, or surgical resection of a subject. The sample may be isolated from a human patient at one or more time points, such that at least one tissue sample is isolated from each time point from the same patient. The sample may be isolated from multiple spatial locations from the same patient at the same time point. The sample may include a single cell or multiple cells or fragments of cells or an aliquot of body fluid, taken from a subject, by means including, but not limited to, venipuncture, excretion, biopsy, needle aspirate, lavage sample, scraping, surgical incision, or intervention or other methods known in the art. The sample can be obtained by the subject or by a third party, e.g., a medical professional. Examples of medical professionals include physicians, emergency medical technicians, nurses, first responders, psychologists, medical physics personnel, nurse practitioners, surgeons, dentists, and any other obvious medical professional as would be known to one skilled in the art. In certain embodiments, a sample can include peripheral blood cells, isolated leukocytes, or RNA extracted from peripheral blood cells or isolated leukocytes.

It must also be noted that as used herein and in the appended claims, the singular forms “a”, “an”, and “the” include plural reference unless the context clearly dictates otherwise.

As used in this document, terms “comprise,” “have,” and “include” and their conjugates, as used herein, mean “including but not limited to.” While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used herein, the term “treatment” or “treating” is defined as the application or administration of a therapeutic agent or mixture of agents, e.g., a compound disclosed herein (alone or in combination with another pharmaceutical agent), to a patient, or application or administration of a therapeutic agent or mixture of agents to a tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, sublingual, pulmonary and topical administration. The compounds can also be a pharmaceutically acceptable salt of the compounds described herein.

As used herein, the terms “effective amount,” “pharmaceutically effective amount” and “therapeutically effective amount” refer to a nontoxic but sufficient amount of an agent to provide the desired biological result. In certain embodiments, the phrase “effective amount” or “therapeutically effective amount,” as used herein, refers to an amount that is sufficient or effective to prevent or treat (delay or prevent the onset of, prevent the progression of, inhibit, decrease or reverse) a disease or condition associated with the Sigma1 receptor, including alleviating symptoms of such diseases. That result may be reduction and/or alleviation of the signs, symptoms, or causes of a disease, or any other desired alteration of a biological system.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the patient. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl. In certain embodiments, a (C₁-C₆) alkyl is ethyl, methyl, isopropyl, isobutyl, n-pentyl, n-hexyl and cyclopropylmethyl.

As used herein, the term “substituted alkyl” means alkyl as defined above, substituted by one, two or three substituents selected from the group consisting of halogen, —OH, alkoxy, —NH₂, —N(CH₃)₂, —C(═O)OH, trifluoromethyl, —CN, —C(═O)O(C₁-C₄)alkyl, —C(═O)NH₂, —SO₂NH₂, —C(═NH)NH₂, and —NO₂, such as containing one or two substituents selected from halogen, —OH, alkoxy, —NH₂, trifluoromethyl, —N(CH₃)₂, and —C(═O)OH, such as selected from halogen, alkoxy and —OH. Examples of substituted alkyls include, but are not limited to, 2,2-difluoropropyl, 2-carboxycyclopentyl and 3-chloropropyl.

As used herein, the term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. In certain embodiments, the alkoxy is C₁-C₃ alkoxy, such as ethoxy and methoxy.

As used herein, the term “halo” or “halogen” alone or as part of another substituent means, unless otherwise stated, a fluorine, chlorine, bromine, or iodine atom, or, fluorine, chlorine, or bromine, or, fluorine or chlorine.

As used herein, the term “cycloalkyl” refers to a mono cyclic or polycyclic non-aromatic radical, wherein each of the atoms forming the ring (i.e. skeletal atoms) is a carbon atom. In one embodiment, the cycloalkyl group is saturated or partially unsaturated. In other embodiments, the cycloalkyl group is fused with an aromatic ring. Cycloalkyl groups include groups having from 3 to 10 ring atoms. Illustrative examples of cycloalkyl groups include, but are not limited to, the following:

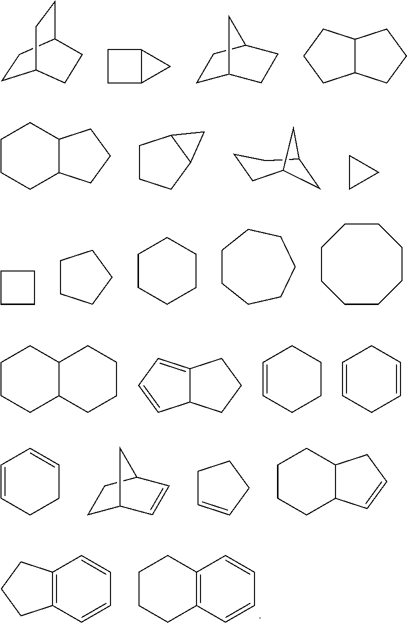

Monocyclic cycloalkyls include, but are not limited to, cyclopropyl, cyclobutyl, cyclopentyl, cyclohexyl, cycloheptyl, and cyclooctyl. Dicyclic cycloalkyls include, but are not limited to, tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle as defined herein, which contains at least one carbon carbon double bond or one carbon carbon triple bond.

As used herein, the term “heterocycloalkyl” or “heterocyclyl” refers to a heteroalicyclic group containing one to four ring heteroatoms each selected from O, Sand N. In one embodiment, each heterocycloalkyl group has from 4 to 10 atoms in its ring system, with the proviso that the ring of said group does not contain two adjacent O or S atoms. In other embodiments, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups are:

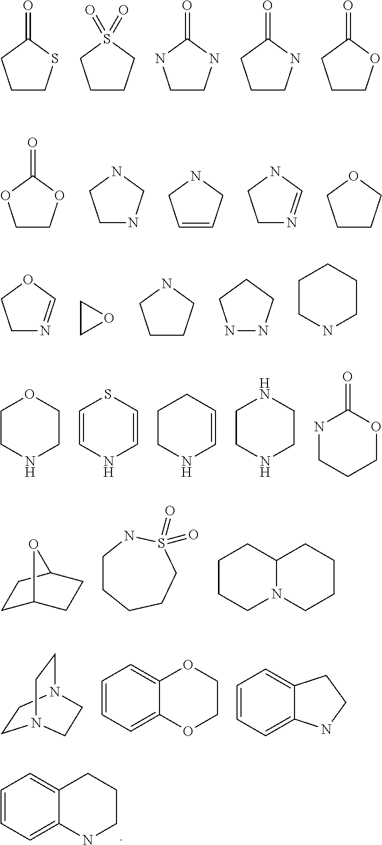

Examples of non-aromatic heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e. having (4n+2) delocalized a (pi) electrons, where n is an integer.

As used herein, the term “aryl,” employed alone or in combination with other terms, means, unless otherwise stated, a carbocyclic aromatic system containing one or more rings (such as one, two or three rings), wherein such rings may also be attached together in a pendent manner, such as a biphenyl, or may be fused, such as naphthalene. Examples of aryl groups include phenyl, anthracyl, and naphthyl. Non-limiting examples are phenyl and naphthyl. In certain embodiments, the aryl is a phenyl.

As used herein, the term “aryl-(C₁-C₃)alkyl” means a functional group wherein a one- to three-carbon alkylene chain is attached to an aryl group, e.g., 13 CH₂CH₂-phenyl. In certain embodiments, the group is aryl-CH₂— and aryl-CH(CH₃)—. The term “substituted aryl-(C₁-C₃)alkyl” means an aryl-(C₁-C₃)alkyl functional group in which the aryl group is substituted. In certain embodiments, the group is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. In certain embodiments, the group is heteroaryl-(CH₂)—. The term “substituted heteroaryl-(C₁-C₃)alkyl” means a heteroaryl-(C₁-C₃)alkyl functional group in which the heteroaryl group is substituted. In certain embodiments, the group is substituted heteroaryl-(CH₂)—.

As used herein, the term “heteroaryl” or “heteroaromatic” refers to a heterocycle having aromatic character. A polycyclic heteroaryl may include one or more rings that are partially saturated. Examples include the following moieties:

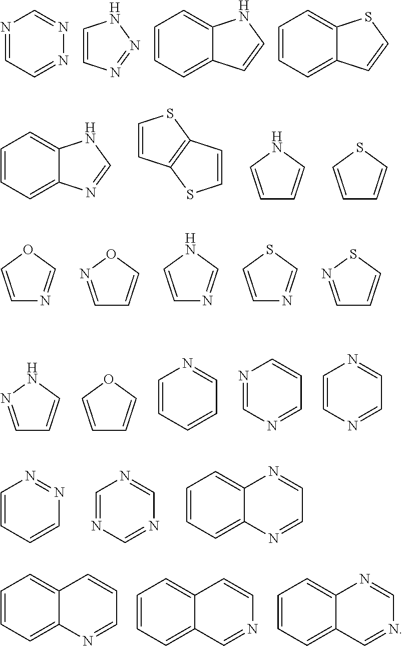

Examples of heteroaryl groups also include pyridyl, pyrazinyl, pyrimidinyl (particularly 2- and 4-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In other embodiments, the substituents vary in number between one and three. In yet another embodiment, the substituents vary in number between one and two.

As used herein, the term “optionally substituted” means that the referenced group may be substituted or unsubstituted. In certain embodiments, the referenced group is optionally substituted with zero substituents, i.e., the referenced group is unsubstituted. In other embodiments, the referenced group is optionally substituted with one or more additional group(s) individually and independently selected from groups described herein.

In certain embodiments, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S-alkyl, S(═O)₂alkyl, —C(═O)NH[substituted or unsubstituted alkyl, or substituted or unsubstituted phenyl], —C(═O)N[H or alkyl]₂, —OC(═O)N[substituted or unsubstituted alkyl]₂, —NHC(═O)NH[substituted or unsubstituted alkyl, or substituted or unsubstituted phenyl], —NHC(═O)alkyl, —N[substituted or unsubstituted alkyl]C(═O)[substituted or unsubstituted alkyl], —NHC(═O)[substituted or unsubstituted alkyl], —C(OH)[substituted or unsubstituted alkyl]₂, and —C(NH₂)[substituted or unsubstituted alkyl]₂. In other embodiments, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCH₂CH₃, —OCH(CH₃)₂, —OCF₃, —OCH₂CF₃, —S(═O)₂—CH₃, —C(═O)NH₂, —C(═O)—NHCH₃, —NHC(═O)NHCH₃, —C(═O)CH₃, and —C(═O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic. In certain embodiments, the chain is straight.

Ranges: various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range and also the endpoints of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### EXPERIMENTAL EXAMPLES

Embodiments are now described with reference to the following Examples. These Examples are provided for the purpose of illustration only, and the embodiments are not limited to these Examples, but rather encompasses all variations that are evident as a result of the teachings provided herein.

### Example 1

This example provides evidence for connecting gene expression data organized in the context of gene sets, e.g., biological pathways, with efficacy of one or more therapeutic agents. A gene set expression profile, also referred to as a gene set-expression signature, was determined for two or more genes in a target cell. The gene set expression profile of the target cell was compared to one or more gene set expression profiles for one or more reference cells, or a panel of reference cells, wherein the panel comprises cells from more than two different cell types. The expression levels of the six (6) human genes listed in Tables 2-3 were analyzed:

TABLE 2

| Human Gene | HUGO Symbol | Entrez GeneID |
|---|---|---|
| 1 | CDH1 | 999 |
| 2 | CREB3L4 | 148327 |
| 3 | PIK3C2B | 5287 |
| 4 | RNF43 | 54894 |
| 5 | SREBF1 | 6720 |
| 6 | ZMYM2 | 7750 |

The sequences of the genes are hereby incorporated by reference in their entirety.

In certain embodiments, methods are provided for connecting gene expression data organized in the context of gene sets, e.g., biological pathways, with efficacy of one or more therapeutic agents. In certain embodiments, the methods comprise determining a gene set expression profile, also referred to as a gene set-expression signature, for two or more genes in a target cell. The gene set expression profile of the target cell can be compared to one or more gene set expression profiles for one or more reference cells, or a panel of reference cells, wherein the panel comprises cells from more than two different cell types.

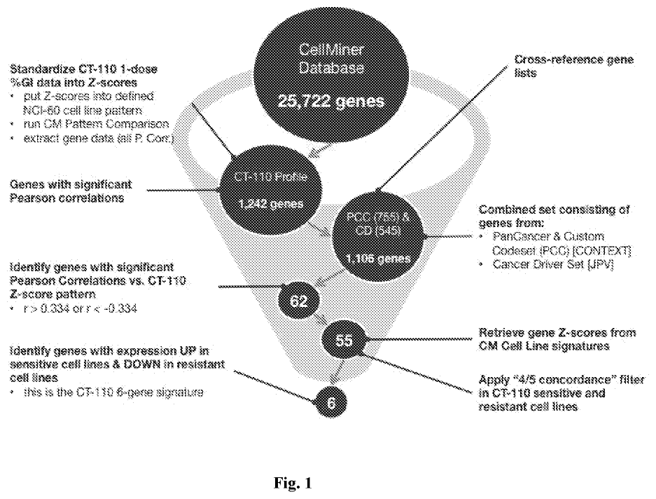

As in FIG. 1, Compound CT-110 was used in single-dose (10 μM) in vitro growth inhibition assays of the NCI-60 cell lines. The NCI-60 panel contains 60 diverse human cancer cell lines screened with more than 100,000 chemical compounds for anticancer activity since 1990 by the Developmental Therapeutics Program (DTP). See Huang, et al, 2005, Pharmacogenomics J. 5:381-99; Staunton, et al., 2001, PNAS USA 98(19):10787-92; Covell, et al., 2007, Mol. Cancer Ther. 6:2261-70; Potti, et al., 2006, Nat. Med. 12(11): 1294-300.

The inhibition sensitivity profile was used to identify genes with similar expression patterns in NCI-60 cell lines. To facilitate the objective, the CellMiner™ database Pattern Comparison tool was used to query the expression profiles of nearly 26,000 genes (FIG. 1).

Standardization of NCI-60 Inhibition Data into Z-Scores.

NCI-60 cell line percent growth data obtained after culture in the presence of CT-110 were converted to percent growth inhibition by the formula 100−% Growth. A Z-score for each cell line data point was calculated using the formula (X-value minus 60-element array average)/(Array standard deviation). Z-scores are associated with a normal distribution and are used (1) to indicate where a score lies relative to the entire data set, above or below the mean, and (2) to compare scores derived from different normal distributions.

Narrowing the 25K+CellMiner Genes to a CT-110 Inhibition Signature Set of 6 Genes.

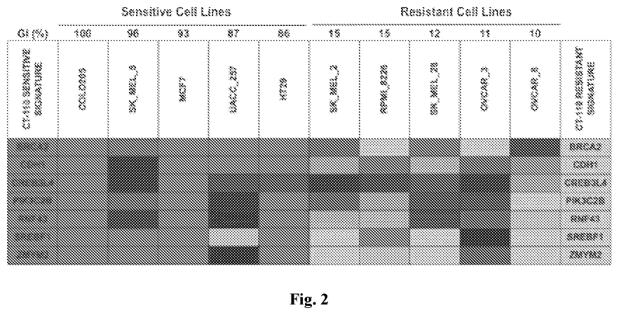

Pattern comparison searching yielded a list of 1,080 genes having significant Pearson correlations (r>0.334 or r<−0.334) to the NCI-60 Z-score profile of CT-110. Cross-referencing this profile-matching list with 1,106 cancer-relevant genes comprising a 755-gene PanCancer and Custom Codeset, supplemented with 545 “Cancer Driver” genes (Integrative OncoGenomics, BROAD Institute TARGET Database v3, Targeted Therapies for Cancer), reduced the number of genes to fifty (50). NCI-60-based Z-score profiles for these 50 genes were obtained using the CellMiner “Cell Line Signature Analysis” query. As in FIG. 2, for the five cell lines most sensitive (COLO205, SK-MEL-5, MCF7, UACC-257, HT29) and the 5 cell lines most resistant (SK-MEL-2, RPMI-8226, SK-MEL-28, OVCAR-3, OVCAR-8) to Sigma1 inhibition by CT-110, a “4 out of 5” concordance approach was utilized to further filter the genes. Z-scores, reflective of gene expression level and directionality, were used to identify forty-five (45) genes out of the 57 (Table 3) that had expression either up or down in at least 4 out of the 5 CT-110-sensitive or resistant cell lines (assessed independently). Since the overall objective of these analyses is to discover a small set of genes that could be used as a repertoire of potential biomarkers of CT-110-mediated inhibition of Sigma1, from among the 45 genes was identified a set of six (6) that displayed concomitant “up” expression in the five CT-110-sensitive cell lines and “down” expression in the five CT-110-resistant cell lines. These six genes (CDH1, CREB3L4, PIK3C2B, RNF43, SREBF1 and ZMYM2) are the end result of the rational approach to establish a focused set of genes, with expression profiles reflecting the growth inhibition profile of CT-110 in sensitive and resistant cancer cell lines.

TABLE 3
Candidate Z Scores Across 5 Most Sensitive And 5 Most Resistant NCI60 Cell Lines

| Entrez | | NCI-60 Cell Line (Z score) | | | | | | | | | |
|---|---|---|---|---|---|---|---|---|---|---|---|
| ID | Gene | HT29 | UACC257 | MCF7 | SKMEL5 | COLO205 | OVCAR8 | OVCAR3 | SKMEL28 | RPMI8226 | SKMEL2 |
| 91 | ACVR1B | 0.86 | −0.41 | 0.50 | 0.14 | 2.59 | 0.13 | 0.21 | −0.79 | −1.12 | −0.54 |
| 27125 | AFF4 | −0.40 | 0.97 | −0.12 | 0.37 | −1.01 | 0.29 | 0.33 | 1.02 | −2.65 | 1.61 |
| 367 | AR | −0.73 | −0.53 | 1.78 | −0.16 | −0.40 | −0.24 | −0.53 | 0.15 | −0.53 | −0.69 |
| 8938 | BAIAP3 | 1.22 | −1.04 | 1.37 | 0.94 | 1.34 | 0.21 | −0.10 | −0.79 | 0.61 | −0.70 |
| 675 | BRCA2 | 2.08 | 1.08 | 1.88 | 0.70 | 1.91 | −0.13 | −1.09 | −0.40 | −1.15 | −0.33 |
| 51806 | CALML5 | 0.77 | −0.07 | 2.25 | −0.06 | 5.45 | −0.16 | 0.01 | −0.35 | −0.15 | −0.26 |
| 896 | CCND3 | 0.50 | 0.27 | −0.71 | −0.04 | 2.03 | −0.54 | −0.76 | −0.16 | −1.24 | −0.38 |
| 999 | CDH1 | 1.65 | 1.43 | 2.12 | 0.08 | 1.20 | −0.52 | 0.67 | −0.67 | −0.39 | −0.60 |
| 9575 | CLOCK | 0.70 | −0.35 | 0.52 | −0.23 | 0.06 | 0.76 | 0.69 | 0.15 | −1.39 | 1.02 |
| 148327 | CREB3L4 | 0.47 | −0.18 | 3.18 | 0.15 | 1.39 | −0.60 | 0.10 | −0.24 | 0.35 | 0.15 |
| 1499 | CTNNB1 | −0.34 | 2.70 | −0.31 | 2.63 | −0.22 | 0.43 | 0.29 | 0.97 | −1.34 | 1.03 |
| 27123 | DKK2 | −0.17 | 4.50 | −0.16 | 2.38 | −0.26 | −0.23 | 0.51 | 0.45 | 0.22 | 0.23 |
| 54567 | DLL4 | −0.26 | −0.04 | 0.02 | −0.26 | 6.09 | −0.27 | −0.31 | −0.29 | 0.02 | −0.53 |
| 1943 | EFNA2 | 3.14 | −0.46 | 0.69 | 1.27 | 1.10 | 0.21 | −0.26 | −0.41 | −0.40 | −0.90 |
| 1956 | EGFR | 0.63 | −1.24 | −1.12 | −0.39 | −0.54 | 0.85 | 0.16 | −1.20 | −1.13 | −1.30 |
| 1999 | ELF3 | 1.75 | −0.71 | 0.47 | −0.75 | 2.01 | −0.68 | 0.98 | −0.74 | −0.55 | −0.88 |
| 1969 | EPHA2 | 1.09 | −0.09 | −1.40 | −0.93 | −0.43 | 0.37 | 1.90 | −1.49 | −1.56 | −0.39 |
| 2064 | ERBB2 | 0.91 | −0.41 | 0.61 | −0.98 | 1.29 | 0.13 | −0.06 | −0.42 | −1.48 | −0.97 |
| 2065 | ERBB3 | 1.25 | 0.81 | 1.26 | −0.32 | 1.00 | 0.64 | 0.23 | 1.08 | −0.99 | 1.67 |
| 2066 | ERBB4 | −0.33 | −0.42 | 1.52 | −0.17 | −0.28 | −0.34 | 1.65 | −0.34 | −0.24 | −0.38 |
| 54855 | FAM46C | −0.44 | 0.05 | −0.06 | −0.44 | −0.05 | −0.22 | 0.00 | −0.05 | 4.51 | 0.33 |
| 9965 | FGF19 | 2.91 | −0.36 | −0.23 | −0.23 | 3.22 | −0.28 | −0.13 | −0.41 | −0.09 | −0.46 |
| 2316 | FLNA | −0.51 | −0.19 | −1.43 | −2.10 | −2.83 | 0.12 | 0.15 | −0.26 | −1.88 | 0.73 |
| 10468 | FST | −0.53 | −0.61 | −0.52 | −0.49 | −0.52 | −0.50 | 2.15 | −0.52 | −0.62 | −0.32 |
| 11211 | FZD10 | −0.33 | −0.17 | 0.02 | −0.59 | 4.16 | −0.18 | −0.19 | −0.18 | −0.28 | −0.81 |
| 4616 | GADD45B | −1.27 | −1.95 | −0.01 | −1.29 | −0.88 | 1.16 | −0.46 | −1.39 | 0.52 | 0.30 |
| 6927 | HNF1A | 0.67 | −0.49 | −0.40 | −0.43 | 3.58 | −0.47 | −0.36 | −0.32 | −0.05 | −0.95 |
| 3486 | IGFBP3 | −1.01 | −0.49 | −0.92 | −0.55 | 0.03 | 0.59 | 1.00 | −0.51 | 1.09 | −0.72 |
| 53832 | IL20RA | 1.58 | −0.35 | −0.28 | −0.31 | 4.38 | −0.42 | 1.44 | −0.34 | −0.29 | −0.41 |
| 58985 | IL22RA1 | 2.97 | −0.66 | −0.42 | −0.16 | 3.39 | −0.35 | −0.06 | −0.46 | −0.42 | −0.53 |
| 3664 | IRF6 | 2.84 | −0.59 | 0.98 | −0.52 | 1.88 | −0.56 | 1.15 | −0.54 | −0.48 | −0.63 |
| 4052 | LTBP1 | −0.90 | −0.08 | 0.76 | −0.51 | −0.97 | −0.81 | 2.25 | −0.83 | −0.73 | 0.16 |
| 4233 | MET | 1.22 | 0.92 | −0.71 | 1.22 | −0.29 | −0.47 | −0.64 | −0.90 | −1.89 | −0.34 |
| 200958 | MUC20 | 1.70 | −0.52 | −0.15 | −0.48 | 3.12 | −0.35 | 0.43 | −0.55 | 0.35 | −0.44 |
| 4915 | NTRK2 | −0.46 | −0.06 | −0.14 | −0.30 | 0.10 | −0.39 | 0.46 | 0.22 | 0.19 | −0.25 |
| 5077 | PAX3 | −0.43 | 1.91 | −0.35 | 0.31 | −0.43 | −0.44 | −0.35 | 2.46 | −0.42 | 1.76 |
| 5105 | PCK1 | −0.35 | −0.41 | −0.09 | −0.30 | 4.44 | −0.25 | −0.05 | −0.18 | 0.05 | −0.38 |
| 5133 | PDCD1 | −0.53 | −0.11 | −0.56 | 0.81 | −2.19 | 1.13 | 0.87 | 1.22 | 1.06 | 2.51 |
| 5159 | PDGFRB | −0.54 | −0.73 | −0.44 | −0.02 | 0.16 | 0.99 | −0.33 | −0.47 | −0.43 | −0.60 |
| 5287 | PIK3C2B | 2.14 | 0.08 | 1.50 | 0.61 | 2.07 | −1.09 | 0.39 | −0.19 | −0.61 | −0.31 |
| 5320 | PLA2G2A | 0.04 | −0.38 | −0.02 | −0.16 | 7.08 | −0.10 | −0.03 | −0.26 | −0.20 | −0.11 |
| 255189 | PLA2G4F | 0.82 | −0.62 | 0.99 | −0.35 | 4.96 | −0.33 | −0.17 | −0.20 | 0.02 | −0.70 |
| 10891 | PPARGC1A | −0.50 | 2.76 | −0.56 | 4.11 | 1.43 | −0.50 | 0.91 | −0.42 | −0.45 | −0.55 |
| 11191 | PTEN | 0.45 | −0.09 | 0.14 | 0.00 | −0.61 | 0.84 | −0.06 | −0.11 | −0.07 | −0.35 |
| 54894 | RNF43 | 2.69 | 0.04 | 1.14 | 0.20 | 2.16 | −0.75 | −0.31 | 0.37 | −0.68 | −0.67 |
| 9869 | SETDB1 | 0.58 | −0.06 | 3.11 | 0.11 | 0.14 | −1.48 | 0.14 | −0.85 | 0.41 | −0.31 |
| 10110 | SGK2 | 2.48 | −0.28 | −0.67 | −0.66 | 2.93 | −0.63 | −0.61 | −0.47 | −0.55 | −0.72 |
| 10290 | SIGMAR1 | 0.54 | 0.67 | −4.19 | 0.15 | 0.76 | −0.39 | −1.19 | 0.40 | 1.77 | 0.33 |
| 4089 | SMAD4 | −1.16 | 0.53 | −0.50 | 0.30 | −3.08 | 0.86 | −0.35 | −0.31 | 0.76 | 0.87 |
| 6707 | SPRR3 | 0.06 | 0.21 | −0.10 | −0.29 | 4.32 | −0.10 | −0.11 | 0.08 | −0.25 | −0.30 |
| 6720 | SREBF1 | 0.82 | −0.73 | 1.68 | 0.80 | 2.18 | −1.08 | −0.01 | −1.17 | 1.68 | −0.93 |
| 7113 | TMPRSS2 | 1.46 | −0.48 | 1.03 | −0.46 | 3.56 | −0.41 | −0.22 | −0.26 | 0.12 | −0.59 |
| 8794 | TNFRSF10C | 0.32 | −0.38 | −0.83 | −0.33 | 2.92 | −0.45 | −0.08 | −0.69 | −0.58 | −0.19 |
| 7481 | WNT11 | 0.49 | −0.23 | −0.18 | −0.31 | 4.62 | −0.25 | 0.39 | −0.24 | 0.04 | −0.69 |
| 27033 | ZBTB32 | −0.23 | −0.03 | 0.00 | −0.23 | −0.11 | −0.06 | 0.04 | −0.37 | 0.15 | −0.38 |
| 7750 | ZMYM2 | 1.22 | 0.03 | 1.94 | 0.87 | 1.56 | −0.92 | 0.54 | −1.02 | −0.74 | −0.80 |
| 79755 | ZNF750 | 0.02 | −0.02 | 1.08 | −0.62 | 4.75 | −0.34 | 0.56 | −0.24 | −0.26 | 0.00 |

### Prevalence of Gene Signature in Patient Sample Database

It has been established, by analyses ensuing from CellMiner™ Pattern Comparison, a 7-gene set and a 3-gene core signature that are indicative of specific Sigma1 inhibition by compound, CT-110, in the 60-element NCI cancer cell line panel (NCI-60). It was next sought to identify additional cancer cell lines that exhibit similar gene expression profiles to these signature genes in order to (1) assess the validity of the signature, and (2) to identify other potential cancer indications in which the gene signature is also present. As a step toward achieving this goal, the published Affymetrix mRNA expression data generated from the 1000-plus cancer cell lines of the Broad-Novartis Cancer Cell Line Encyclopedia (CCLE) was investigated

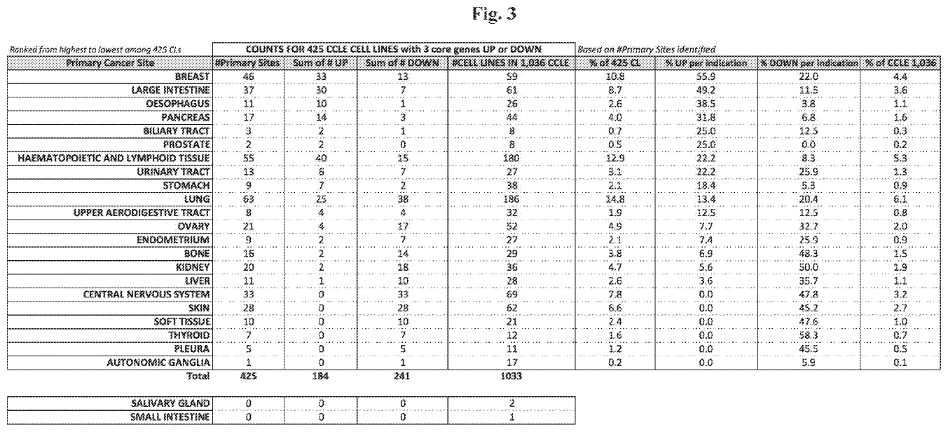

Affymetrix Human Genome U133 Plus 2.0 GeneChip® array data for 1,036 of the 1,046 CCLE cell lines were downloaded as a single 63.4 MB file* from Broad Institute's website (“Browse Data” tab), and accessed using GENE-E, a JAVA-based, high-performance visualization and analysis tool for RNAi and gene expression data. Raw Affymetrix CEL files had been converted to a single value for each probe set using Robust Multi-array Average (RMA) and normalized using quantile normalization; therefore, each gene in the analysis consisted of a single marker (FIG. 2). Gene marker expression intensities across the 1,036 cell lines were standardized into Z-scores, within Excel, to facilitate comparisons between genes and cell lines. With particular interest in the 3-gene core signature (CREB3L4, PIK3C2B, SREBF1), the CCLE cell lines which had all three genes expressed concomitantly up or down were quantified. Four hundred and twenty-five (425) cell lines, representing 22 primary cancer sites of the 24 sites comprising CCLE, were identified that fit these criteria (FIG. 3). Only two primary cancer sites (salivary gland, small intestine), consisting of 3 cell lines, did not meet the expression criteria of all 3 genes up or all 3 genes down.

### In Vitro Prospective Validation

Identifying Sigma1 Sensitive Cell Lines.

Affymetrix Human Genome U133 Plus 2.0 GeneChip® array data for 350 cell lines were downloaded as a single 25.4 MB file from Broad Institute Cancer Cell Line Encyclopedia (CCLE) and accessed using GENE-E, a JAVA-based, high-performance visualization and analysis tool for RNAi and gene expression data. Raw Affymetrix CEL files had been converted to a single value for each probe set using Robust Multi-array Average (RMA) and normalized using quantile normalization; therefore, each gene in the analysis consisted of a single marker. Gene marker expression intensities across the 350 cell lines were standardized into Z-scores, within Excel, to facilitate comparisons between genes and cell lines. We identified 18 cell lines (Table 2) having five of the Sigma1 Signature genes concomitantly up regulated, denoting potential sensitivity to Sigma1 inhibition. 21 cell lines (Table 2) were identified as having five of the Sigma1 Signature genes concomitantly down regulated, denoting potential resistance to Sigma1 inhibition.

Procedure for Evaluating Sigma1 Inhibition of Cell Proliferation.

Cells are harvested from exponential phase cultures, counted and plated in 96 well flat-bottom microtiter plates at a cell density depending on the cell line's growth rate (4,000 and 30,000 cells for solid tumor cell lines, 10,000 to 60,000 for hematological cancer cell lines). After a 24 hour recovery period, 10 μL of culture medium (four control wells/plate) or of culture medium with test compounds are added at increasing concentrations (0.001, 0.0032, 0.01, 0.032, 0.1, 0.32, 1, 3.2, 10, 31.6 μM) by a liquid handling robotic system and treatment is continued for 72 h. Compounds are applied in half-log increments at 10 concentrations in duplicate. CELLTITER-BLUE® assay (# G8081, Promega) is used for read-out. After treatment of cells, 20 μl/well CELLTITER-BLUE® reagent is added. After incubation of up to 4 hours, fluorescence (FU) is measured by using the Enspire Multimode Plate Reader (excitation λ=531 nm, emission λ=615 nm). Sigmoidal concentration-response curves are fitted to the data points (T/C values) obtained for each cell line using 4-parameter non-linear curve fit (Oncotest Warehouse Software). IC₅₀ values are reported as relative IC₅₀ values, being the concentration of test compound that give a response (inhibition of viability) half way between the top and bottom plateau of the sigmoidal concentration-response curve (inflection point of the curve), or as absolute IC₅₀ values, being the concentration of test compound at the intersection of the concentration-response curves with T/C=50%. For calculation of mean IC₅₀ values the geometric mean is used. Results are presented as mean graph plots or heat maps (individual IC₅₀ values relative to the geometric mean IC50 value) over all cell lines as tested.

IC₅₀ values below 10 μM in the CellTiter-Blue® assay are considered sensitive to Sigma1 inhibition.

Predicted Sigma1 response was confirmed in 30 of 39 cell lines overall. There was concordance between predicted and actual Sigma1 inhibitor response in 12 of 18 predicted sensitive cell lines. There was concordance between predicted and actual Sigma1 inhibitor response in 18 of 21 predicted resistant cell lines.

TABLE 4

| | | Predicted | Actual | |
|---|---|---|---|---|
| Primary Site | Cell Line | Response | Response | Concordance |
| Breast | BT474 | Sensitive | Sensitive | Yes |
| Breast | MCF7 | Sensitive | Sensitive | Yes |
| Breast | MDAMB231 | Resistant | Resistant | Yes |
| Breast | MDAMB453 | Sensitive | Sensitive | Yes |
| Breast | T47D | Sensitive | Sensitive | Yes |
| Breast | ZR751 | Sensitive | Sensitive | Yes |
| Central Nervous | DAOY | Resistant | Resistant | Yes |
| Central Nervous | M059K | Resistant | Resistant | Yes |
| Central Nervous | SNB19 | Resistant | Resistant | Yes |
| Central Nervous | SW579 | Resistant | Resistant | Yes |
| Central Nervous | U87MG | Resistant | Resistant | Yes |
| Esophagus | KYSE150 | Sensitive | Resistant | No |
| Kidney | CAKI1 | Resistant | Sensitive | No |
| Kidney | 786O | Resistant | Resistant | Yes |
| Large Intestine | DLD1 | Sensitive | Sensitive | Yes |
| Large Intestine | RKO | Resistant | Resistant | Yes |
| Liver | HLE | Resistant | Resistant | Yes |
| Liver | SNU423 | Resistant | Sensitive | No |
| Lung | CALU1 | Resistant | Resistant | Yes |
| Lung | NCIH226 | Resistant | Resistant | Yes |
| Hematopoietic | HUT78 | Resistant | Resistant | Yes |
| Hematopoietic | SUDHL1 | Resistant | Resistant | Yes |
| Ovary | OVCAR8 | Resistant | Resistant | Yes |
| Ovary | SKOV3 | Resistant | Resistant | Yes |
| Pancreas | HPAFII | Sensitive | Resistant | No |
| Pancreas | YAPC | Sensitive | Sensitive | Yes |
| Prostate | VCAP | Sensitive | Resistant | No |
| Skin | SKMEL2 | Resistant | Resistant | Yes |
| Soft Tissue | HT1080 | Resistant | Resistant | Yes |
| Stomach | IM95 | Sensitive | Sensitive | Yes |
| Stomach | MKN45 | Sensitive | Resistant | No |
| Stomach | NUGC4 | Sensitive | Sensitive | Yes |
| Stomach | OCUM1 | Sensitive | Sensitive | Yes |
| Stomach | SNU16 | Sensitive | Resistant | No |
| Thyroid | SW579 | Resistant | Sensitive | No |
| Upper Aerodigestive | CAL27 | Sensitive | Sensitive | Yes |
| Upper Aerodigestive | FADU | Sensitive | Resistant | No |
| Upper Aerodigestive | CAL33 | Sensitive | Sensitive | Yes |
| Urinary Tract | T24 | Resistant | Resistant | Yes |

### In Vivo Prospective Validation

Sensitive (BT474) and resistant (MDAMB231) cell lines were selected for in vivo analysis based on concordant response between gene expression profile (Table 3) and in vitro response (Table 2).

TABLE 5

| HUGO Symbol | BT474 | MDAMB231 |
|---|---|---|
| CDH1 | Increase | Decrease |
| CREB3L4 | Increase | Decrease |
| PIK3C2B | Increase | Decrease |
| RNF43 | Increase | Decrease |
| SREBF1 | Increase | Decrease |
| ZMYM2 | Increase | Decrease |

BT474 cells (ATCC, Manassas, Va., cat # HTB-20) were implanted orthotopically (2×10⁶ per mouse) as a mixture in Matrigel (50%) into the mammary fat pad of female SCID Beige mice (10 mice per treatment arm) and grown to a volume of about 125 mm³ prior to compound administration. CT-110 was dosed alone at 10 mg/kg ip, once every two days (ip, q2d) in 1% (weight/volume) sodium carboxymethyl cellulose/0.5% (volume/volume) Tween-80. A control group received vehicle alone (1% (weight/volume) sodium carboxymethyl cellulose/0.5% (volume/volume) Tween-80ip q2d). All drug treatment was stopped on Day 30.

MDAMB231 cells (ATCC, Manassas, Va., cat # HTB-26) were implanted orthotopically (2×10⁶ per mouse) as a mixture in Matrigel (50%) into the mammary fat pad of female SCID Beige mice (10 mice per treatment arm) and grown to a volume of about 75 mm³ prior to compound administration. CT-110 was dosed alone at 10 mg/kg ip, once every two days (ip, q2d) in 1% (weight/volume) sodium carboxymethyl cellulose/0.5% (volume/volume) Tween-80. A control group received vehicle alone (1% (weight/volume) sodium carboxymethyl cellulose/0.5% (volume/volume) Tween-80ip q2d). All drug treatment was stopped on Day 20.

The major endpoint is to assess whether the tumor growth can be delayed or mice can be cured. Tumor sizes are measured twice weekly in two dimensions using a caliper, and the volume is expressed in mm³ using the formula: V=0.5 a×b² where a and b are the long and short diameters of the tumor, respectively. The tumor sizes are then used for the calculations of both T-C and T/C values. T-C is calculated with T as the median time (in days) required for the treatment group tumors to reach a predetermined size (e.g., 1,000 mm³), and C is the median time (in days) for the control group tumors to reach the same size. The T/C value (in percent) is an indication of antitumor effectiveness, T and C are the mean volume of the treated and control groups, respectively, on a given day.

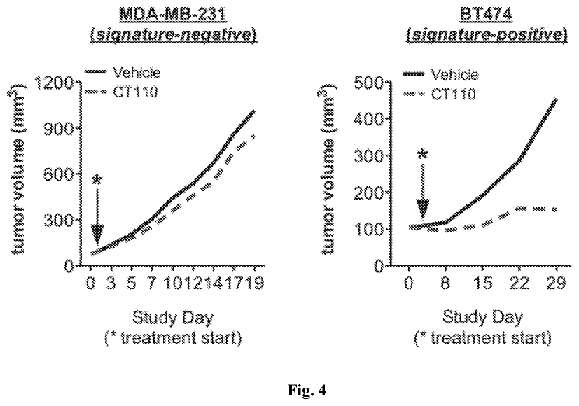

CT-110 significantly suppressed tumor growth (70% T/C) in the predicted sensitive model (BT474) whereas CT-110 had minimal effect on tumor growth (<10% T/C) in the predicted resistant model (MDAMB231) (FIG. 4).

### Example 2: Treatment of Patients

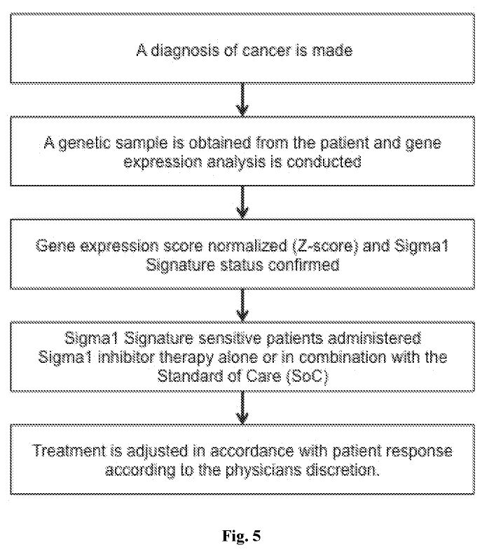

The following example describes how to determine a Sigma1 Signature for predicting response to Sigma1 inhibitors, as exemplified by CT-110, in patients suffering from cancer (FIG. 5). A diagnosis of bladder, breast, central nervous, cervical, colon, esophageal, head and neck, hematopoietic, intestinal, lung, ocular, oral, ovarian, pancreatic, prostate, rectal, renal, skin, soft tissue, stomach, thyroid, urinary tract, or uterine cancer is made.

A genetic sample is obtained from the patient in the form of blood, urine, or tissue.

The sample is processed by the methods provided elsewhere herein, along with NANOSTRING® patent document(s) (such as US20100015607, US20100047924, and US20100112710). Z-scores are tabulated to determine gene expression changes (increase, decrease, no change) of the Sigma1 Signature genes (Table 2) in patient samples.

An increase in Z-scores in at least three of seven Sigma1 Signature genes from patient samples is considered Sigma1 inhibitor sensitive. A decrease in Z-scores in at least three of seven Sigma1 Signature genes from patient samples is considered Sigma1 inhibitor resistant.

If a patient sample is deemed sensitive based upon the Sigma1 Signature profile, then the patient is deemed a suitable candidate for Sigma1 inhibition.

Treatment is adjusted in accordance with patient response and established treatment protocols. Treatment may contain a Sigma1 inhibitor alone or in combination with additional therapies and anticancer agents. Examples of therapies and anticancer agents that can be used in combination with a Sigma1 inhibitor include surgery, radiotherapy (e.g., gamma-radiation, neutron beam radiotherapy, electron beam radiotherapy, proton therapy, brachytherapy, and systemic radioactive isotopes), endocrine therapy, a biologic response modifier (e.g., an interferon, an interleukin, tumor necrosis factor (TNF), hyperthermia and cryotherapy, an agent to attenuate any adverse effect (e.g., an antiemetic), and any other approved chemotherapeutic drug.

The response data associated with the tumor samples tested are obtained from the hospital or clinical laboratory supplying the tumor samples. Clinical response is typically defined in terms of tumor shrinkage, e.g., 30% shrinkage, as determined by suitable imaging technique, e.g., CT Scan. In some cases, human clinical response is defined in terms of time, e.g., progression free survival time. The optimal threshold protein homeostasis signature score for the given tumor type is calculated, as described above. Subsequently, this optimal threshold protein homeostasis signature score is used to predict whether newly tested human tumors of the same tumor type will be response or non-responsive to treatment with a Sigma1 inhibitor.

Various references and patents are disclosed herein, each of which are hereby incorporated by reference for the purpose that they are cited.

From the foregoing, it will be appreciated that various embodiments of the present disclosure have been described herein for purposes of illustration, and that various modifications can be made without departing from the scope and spirit of the present disclosure. Accordingly, the various embodiments disclosed herein are not intended to be limiting.

1. A method of treating cancer in a subject using a Sigma1 inhibitor, the method comprising:
a) measuring the expression levels of at least three genes from a cancer tissue sample that is taken from the subject and contacted with a Sigma1 inhibitor, wherein the at least three genes are selected from the group consisting of CDH1, CREB3L4, PIK3C2B, RNH43, SREBF1, and ZMYM2;
b) calculating a signature score based upon the relative expression levels of the at least three genes, wherein increase in the relative expression levels of the at least three genes as compared to controls identifies the subject as a subject that can be treated for the cancer with a Sigma1 inhibitor; and
c) administering to the identified subject a therapeutically effective amount of a Sigma1 inhibitor.

2. The method of claim 1, wherein the cancer tissue sample is at least one selected from the group consisting of blood, urine, and tissue.

3. The method of claim 1, wherein the Sigma1 inhibitor is selected from the group consisting of
CT-110,
IPAG,
haloperidol,
PB28,
rimcazole,
a compound of Formula (I): 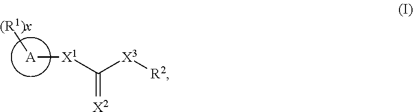
wherein in (I):
ring A is a monocyclic or bicyclic aryl or a monocyclic or bicyclic heteroaryl ring, and wherein the aryl or heteroaryl ring is optionally substituted with 0-4 R¹ groups;
each occurrence of R¹ is independently selected from the group consisting of —C₁-C₆ alkyl, —C₁-C₆ fluoroalkyl, —C₁-C₆ heteroalkyl, F, Cl, Br, I, —CN, —NO₂, —OR³, —SR³, —S(═O)R³, —S(═O)₂R³, —NHS(═O)₂R³, —C(═O)R³, —OC(═O)R³, —CO₂R³, —OCO₂R³, —CH(R³)₂, —N(R³)₂, —C(═O)N(R³)₂, —OC(═O)N(R³)₂, —NHC(═O)NH(R³), —NHC(═O)R³, —NHC(═O)OR³, —C(OH)(R³)₂, and —C(NH₂)(R³)₂;
each occurrence of R² is independently selected from the group consisting of H, C₁-C₆ alkyl, C₁-C₆ heteroalkyl, and —C₁-C₃ alkyl-(C₃-C₆ cycloalkyl), wherein the alkyl, heteroalkyl or cycloalkyl group is optionally substituted with 0-5 R¹ groups, or X³ and R² combine to form a (C₃-C₇) heterocycloalkyl group, optionally substituted with 0-2 R¹ groups;
each occurrence of R³ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X¹ is —CH₂—, —S—, —O— or —(NR²)—;
X² is ═CH₂, ═S, ═O or ═NR²; and
X³ is —S—, —O—, or —NR²—; and
a compound of Formula (II):
R^{A}—R^{B} (II)
wherein in (II):
R^{A} is selected from the group consisting of 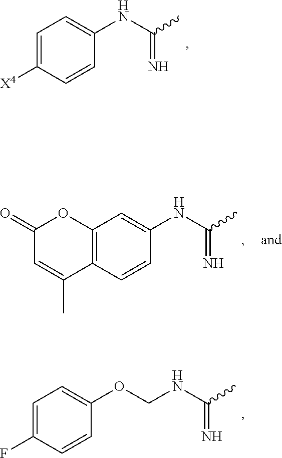
X⁴ is selected from the group consisting of OMe, F, Cl, Br, and I; and
R^{B} is selected from the group consisting of: 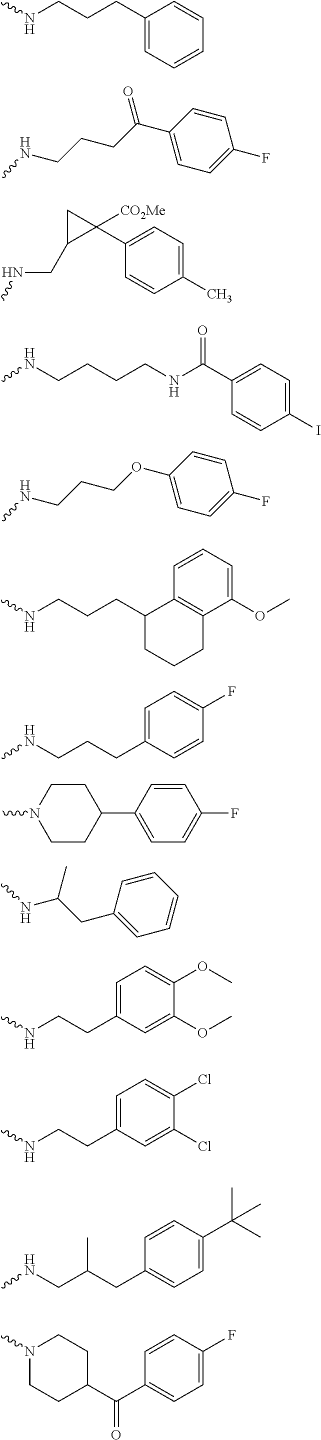 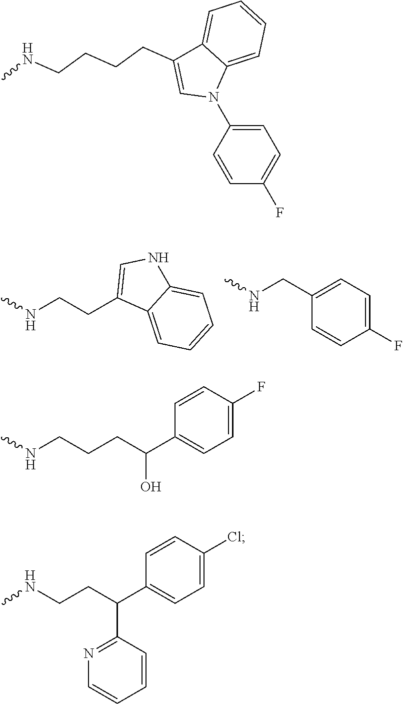
a pharmaceutically acceptable salt or solvate thereof, a N-oxide thereof, and any combinations thereof.

4. The method of claim 3, wherein the at least one compound is a compound of Formula (I-A), or a pharmaceutically acceptable salt or solvate thereof:
R^{A}—R^{B} (I-A),
wherein in (I-A):
R^{A} is selected from the group consisting of 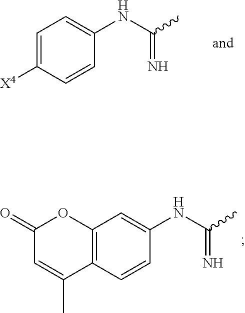
X⁴ is selected from the group consisting of OMe, F, Cl, Br, and I; and
R^{B} is selected from the group consisting of:

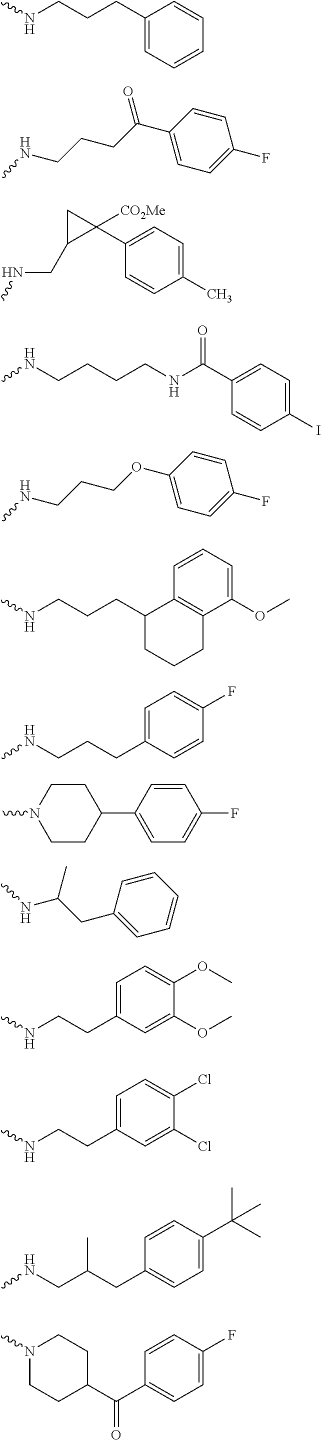

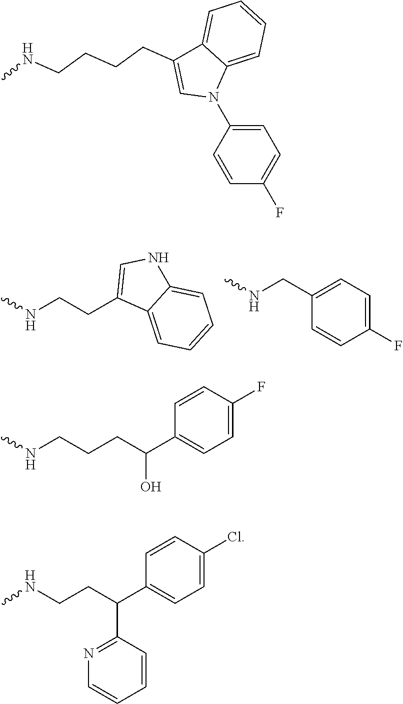

5. The method of claim 4, wherein in the compound of formula (I-A):
R^{A} is 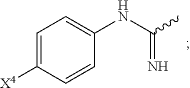
X⁴ is selected from the group consisting of F, Cl, Br, and I; and
R^{B} is selected from the group consisting of:

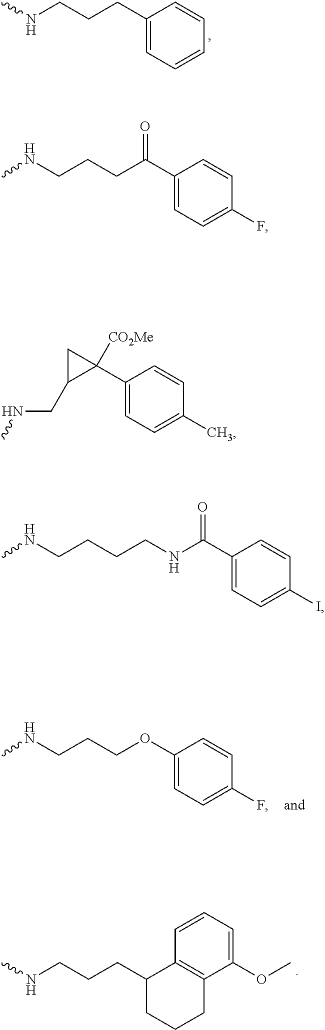

6. The method of claim 4, wherein in the compound of formula (I-A):
R^{A} is 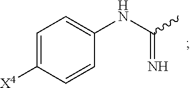
X⁴ is selected from the group consisting of F, Cl, Br, and I; and
R^{B} is selected from the group consisting of:

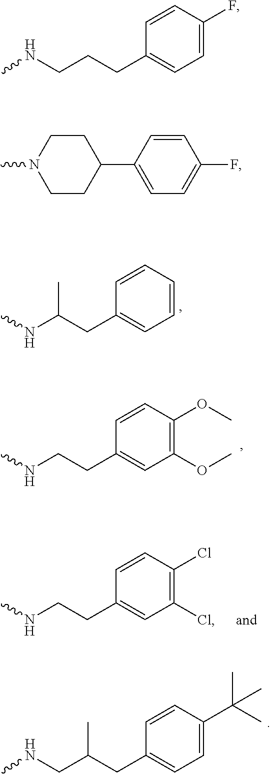

7. The method of claim 4, wherein in the compound of formula (I-A):
R^{A} is 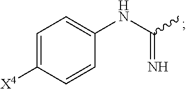
X⁴ is selected from the group consisting of F, Cl, Br, and I; and
R^{B} is selected from the group consisting of:

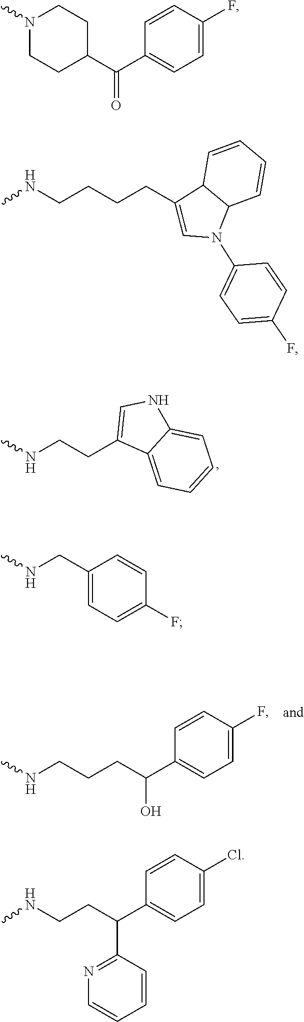

8. The method of claim 3, wherein in the compound of formula (II):
R^{A} is 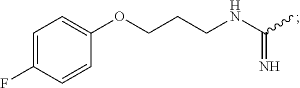
and
R^{B} is selected from the group consisting of:

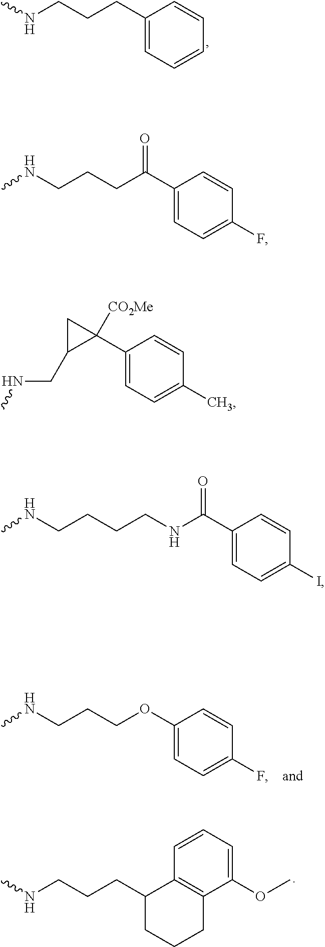

9. The method of claim 3, wherein in the compound of formula (II):
R^{A} is 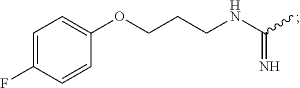
and
R^{B} is selected from the group consisting of:

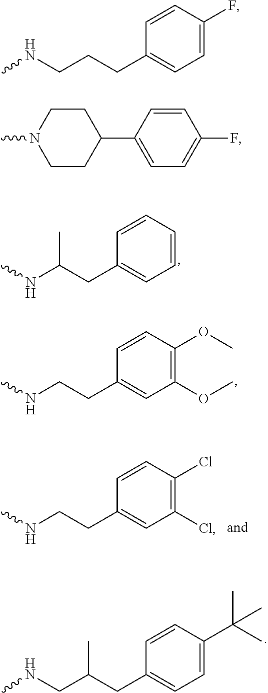

10. The method of claim 3, wherein in the compound of formula (II):
R^{A} is 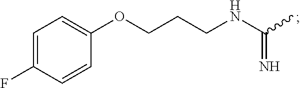
and
R^{B} is selected from the group consisting of:

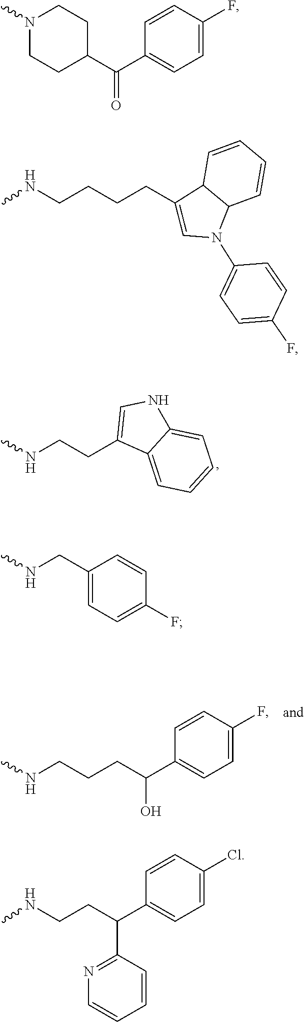

11. The method of claim 4, wherein in the compound of Formula (I-A):
R^{A} is 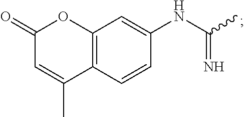
and
R^{B} is selected from the group consisting of:

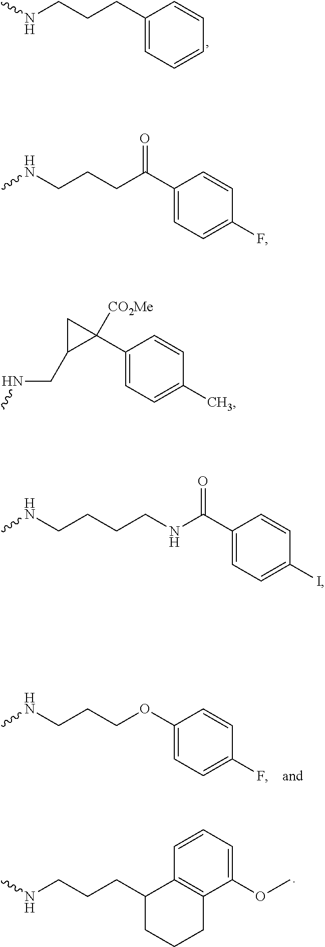

12. The method of claim 4, wherein in the compound of Formula (I-A):
R^{A} is 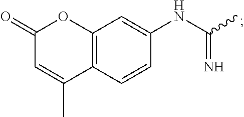
and
R^{B} is selected from the group consisting of:

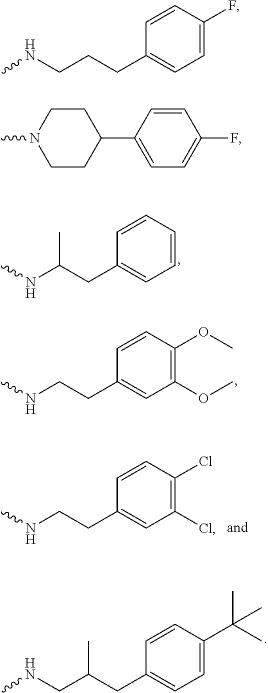

13. The method of claim 4, wherein in the compound of Formula (I-A):
R^{A} is 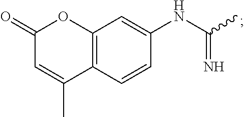
and
R^{B} is selected from the group consisting of:

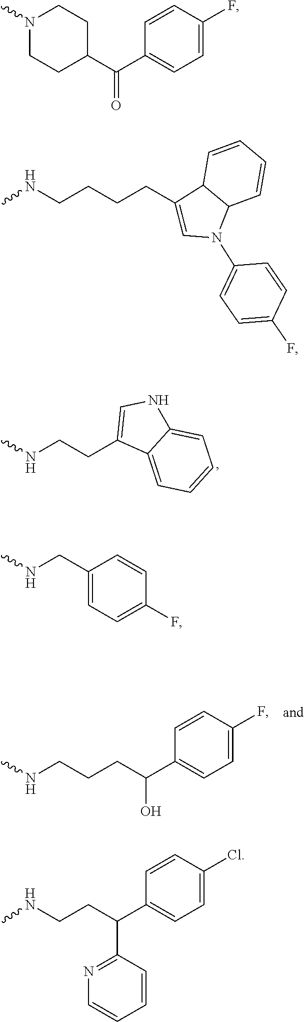

14. The method of claim 3, wherein the at least one compound is a compound of formula (I-B), or a pharmaceutically acceptable salt or solvate thereof: 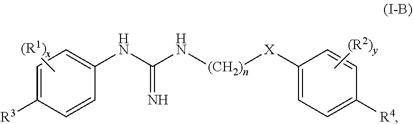
wherein in (I-B):
each occurrence of R¹ and R² is independently selected from the group consisting of —C₁-C₆ alkyl, —C₁-C₆ fluoroalkyl, —C₁-C₆ heteroalkyl, F, Cl, Br, I, —CN, —NO₂, —OR⁵, —SR⁵, —S(═O)R⁵, —S(═O)₂R⁵, —NHS(═O)₂R⁵, —C(═O)R⁵, —OC(═O)R⁵, —CO₂R⁵, —OCO₂R⁵, —CH(R⁵)₂, —N(R⁵)₂, —C(═O)N(R⁵)₂, —OC(═O)N(R⁵)₂, —NHC(═O)NH(R⁵), —NHC(═O)R⁵, —NHC(═O)OR⁵, —C(OH)(R⁵)₂, and —C(NH₂)(R⁵)₂;
R³ is selected from the group consisting of —C₁-C₆ alkyl, —C₁-C₆ fluoroalkyl, —C₁-C₆ alkoxy, F, Cl, Br, and I;
R⁴ is selected from the group consisting of —C₁-C₆ alkyl, —C₁-C₆ alkoxy, F, Cl, Br, and I;
each occurrence of R⁵ is independently selected from the group consisting of H, C₁-C₆ alkyl, C₁-C₆ heteroalkyl, aryl, and —C₁-C₃ alkyl-(C₃-C₆ cycloalkyl), wherein the alkyl, heteroalkyl, aryl, or cycloalkyl group is optionally substituted;
X is selected from the group consisting of CH₂, C═O, and O;
n is an integer from 1-3;
x is an integer from 0-4; and
y is an integer from 0-4.

15. The method of claim 3, wherein the at least one compound is selected from the group consisting of:
1-(3-(4-fluorophenoxy)propyl)-3-(4-iodophenyl)guanidine (Compound A),
1-(3-(4-fluorophenoxy)propyl)-3-(4-methoxyphenyl)guanidine (Compound B),
1-(n-propyl)-3-(4-iodophenyl)guanidine (Compound C),
1-(n-propyl)-3-(4-methoxyphenyl)guanidine (Compound D),
1,3-bis(3-(4-fluorophenoxy)propyl)guanidine (Compound E),
1-(3-(4-fluorophenoxy)propyl)-3-(4-trifluoromethylphenyl)guanidine (Compound F),
1-(3-(4-fluorophenoxy)propyl)-3-(4-chlorophenyl)guanidine (Compound G), and
1-(3-(4-fluorophenoxy)propyl)-3-(4-methyl 1-2-oxo-2H-chromen-7-yl)guanidine) (Compound H),
or a pharmaceutically acceptable salt or solvate thereof, and any combinations thereof.

16. The method of claim 1, wherein the Sigma1 inhibitor is selected from the group consisting of CT-110 (1-(3-(4-fluorophenoxy)propyl)-3-(4-chlorophenyl)guanidine), IPAG (1-(4-Iodophenyl)-3-(2-adamantyl)guanidine), haloperidol (4-[4-(4-Chlorophenyl)-4-hydroxy-1-piperidinyl]-1-(4-fluorophenyl)-1-butanone hydrochloride), PB28 (1-Cyclohexyl-4-[3-(1,2,3,4-tetrahydro-5-methoxy-1-naphthalenyl)propyl]piperazine dihydrochloride), and rimcazole (9-[3-(cis-3,5-Dimethyl-1-piperazinyl)propyl]-9H-carbazole dihydrochloride).

17. The method of claim 1, wherein the subject is further treated with one or more additional therapeutic agents.

18. The method of claim 1, wherein the cancer is at least one selected from the group consisting of bladder, breast, central nervous, cervical, colon, esophagus, head and neck, hematopoietic, intestinal, lung, ocular, oral, ovarian, pancreatic, prostate, rectal, renal, skin, soft tissue, stomach, thyroid, urinary tract, and uterus.

19. The method of claim 1, wherein the cancer is at least one selected from the group consisting of breast and prostate.

20. The method of claim 1, wherein the method comprises measuring at least four genes from a cancer tissue sample, wherein the at least four genes are selected from the group consisting of CDH1, CREB3L4, PIK3C2B, RNH43, SREBF1, and ZMYM2.

21. The method of claim 1, wherein the method comprises measuring at least five genes from a cancer tissue sample, wherein the at least five genes are selected from the group consisting of CDH1, CREB3L4, PIK3C2B, RNH43, SREBF1, and ZMYM2.

22. The method of claim 1, wherein the method comprises measuring each of the six genes selected from the group consisting of CDH1, CREB3L4, PIK3C2B, RNH43, SREBF1, and ZMYM2, from a cancer tissue sample.

23. A method of identifying a tumor as being responsive to treatment with a Sigma1 inhibitor, the method comprising:
a) measuring the expression levels of at least three genes from a cancer tissue sample that is taken from the subject and treated with a Sigma1 inhibitor, wherein the at least three genes are selected from the group consisting of CDH1, CREB3L4, PIK3C2B, RNH43, SREBF1, and ZMYM2;
b) calculating a signature score based upon the relative expression levels of the at least three genes, wherein increase in the relative expression levels of at least three genes selected from the group consisting of CDH1, CREB3L4, PIK3C2B, RNH43, SREBF1, and ZMYM2 as compared to controls identifies a tumor as being responsive to a Sigma1 inhibitor.

24. The method of claim 23, wherein the method further comprises:
a) measuring the relative expression levels of at least four genes from a tumor sample and treated with a Sigma1 inhibitor, wherein at least three out of the at least four genes are selected from the group consisting of CDH1, CREB3L4, PIK3C2B, RNH43, SREBF1, and ZMYM2, and at least one out of the at least four genes is selected from the group consisting of AR, BRCA2, EGFR, ERBB2, ERBB3, FASN, and PTEN; and
b) calculating a signature score based upon the relative expression levels of the at least four genes, wherein increase in the relative expression levels of CDH1, CREB3L4, PIK3C2B, RNH43, SREBF1, ZMYM2, AR, BRCA2, EGFR, ERBB2, ERBB3, or FASN compared to controls increases the signature score, and decrease in the relative expression levels of PTEN compared to control increases the signature score, and wherein the tumor sample is responsive to treatment with a Sigma1 inhibitor if the signature score is higher than a threshold value.

25. A composition comprising polynucleotides specific for at least three genes selected from the group consisting of CDH1, CREB3L4, PIK3C2B, RNH43, SREBF1, and ZMYM2, wherein the polynucleotides comprises a detectable fluorescent label or a non-naturally occurring nucleotide base.

26. The composition of claim 25, wherein at least one of the polynucleotides, or each polynucleotide, comprises at least one non-naturally occurring nucleotide.

27. The composition of claim 25, wherein the polynucleotides are specific for at least one gene selected from the group consisting of CDH1, CREB3L4, PIK3C2B, RNH43, SREBF1, and ZMYM2.

28. The composition of claim 27, wherein at least one polynucleotide comprises at least one non-naturally occurring nucleotide.

29. The composition of claim 25, wherein the composition comprises polynucleotides that are specific for at least 4 genes selected from the group consisting of CDH1, CREB3L4, PIK3C2B, RNH43, SREBF1, and ZMYM2.

30. The composition of claim 25, wherein the polynucleotides are specific for at least 5 genes selected from the group consisting of CDH1, CREB3L4, PIK3C2B, RNH43, SREBF1, and ZMYM2.

31. The composition of claim 25, wherein the polynucleotides are specific for CDH1, CREB3L4, PIK3C2B, RNH43, SREBF1, and ZMYM2.

32. The composition of claim 25, wherein the polynucleotides consist of polynucleotides specific for CDH1, CREB3L4, PIK3C2B, RNH43, SREBF1, and ZMYM2.

33. The composition of claim 25, further comprising a polynucleotide specific for one or two genes selected from the group consisting of AR, BRCA2, EGFR, ERBB2, ERBB3, FASN, and PTEN.

34. The composition of claim 25, further comprising a polynucleotide specific for at least three genes selected from the group consisting of AR, BRCA2, EGFR, ERBB2, ERBB3, FASN, and PTEN.

35. The composition of claim 25, further comprising a polynucleotide specific for at least four genes selected from the group consisting of AR, BRCA2, EGFR, ERBB2, ERBB3, FASN, and PTEN.

36. The composition of claim 25, further comprising a polynucleotide specific for at least five genes selected from the group consisting of AR, BRCA2, EGFR, ERBB2, ERBB3, FASN, and PTEN.

37. The composition of claim 25, further comprising a polynucleotide specific for at least six genes selected from the group consisting of AR, BRCA2, EGFR, ERBB2, ERBB3, FASN, and PTEN.

38. The composition of claim 25, further comprising polynucleotides specific for AR, BRCA2, EGFR, ERBB2, ERBB3, FASN, and PTEN.

39. The composition of claim 25, wherein at least one polynucleotide, or each polynucleotide, comprises a non-naturally occurring nucleotide base.

40. A method of measuring expression levels of at least three genes from a cancer tissue sample that is taken from the subject and contacted with a Sigma1 inhibitor, wherein the at least three genes are selected from a group consisting of CDH1, CREB3L4, PIK3C2B, RNH43, SREBF1, and ZMYM2, wherein the sample is contacted with a composition comprising a plurality of polynucleotides specific for at least three genes selected from the group consisting of CDH1, CREB3L4, PIK3C2B, RNH43, SREBF1, and ZMYM2, under conditions sufficient to measure the expression of at least three of the genes.

41. The method of claim 40, wherein the expression levels of at least four of the genes are measured.

42. The method of claim 40, wherein the expression levels of at least five of the genes are measured.

43. The method of claim 40, wherein the expression levels of the six genes are measured.

44. The method of claim 40, wherein the sample is contacted with a composition comprising polynucleotides specific for 3 genes selected from the group consisting of CDH1, CREB3L4, PIK3C2B, RNH43, SREBF1, and ZMYM2.

45. The method of claim 40, wherein the composition comprises polynucleotides that are specific for at least 4 genes selected from the group consisting of CDH1, CREB3L4, PIK3C2B, RNH43, SREBF1, and ZMYM2.

46. The method of claim 40, wherein the composition comprises polynucleotides that are specific for at least 5 genes selected from the group consisting of CDH1, CREB3L4, PIK3C2B, RNH43, SREBF1, and ZMYM2.

47. The method of claim 40, wherein the composition comprises polynucleotides that are specific for CDH1, CREB3L4, PIK3C2B, RNH43, SREBF1, and ZMYM2.

48. The method of claim 40, wherein the composition consists of polynucleotides that are specific for CDH1, CREB3L4, PIK3C2B, RNH43, SREBF1, and ZMYM2.

49. The method of claim 40, the method further comprising measuring expression of at least one or two genes selected from the group consisting of AR, BRCA2, EGFR, ERBB2, ERBB3, FASN, and PTEN.

50. The method of claim 40, the method further comprising measuring expression of at least three genes selected from the group consisting of AR, BRCA2, EGFR, ERBB2, ERBB3, FASN, and PTEN.

51. The method of claim 40, the method further comprising measuring expression of at least three genes selected from the group consisting of AR, BRCA2, EGFR, ERBB2, ERBB3, FASN, and PTEN.

52. The method of claim 40, the method further comprising measuring expression of at least four genes selected from the group consisting of AR, BRCA2, EGFR, ERBB2, ERBB3, FASN, and PTEN.

53. The method of claim 40, the method further comprising measuring expression of at least five genes selected from the group consisting of AR, BRCA2, EGFR, ERBB2, ERBB3, FASN, and PTEN.

54. The method of claim 40, the method further comprising measuring expression of at least six genes selected from the group consisting of AR, BRCA2, EGFR, ERBB2, ERBB3, FASN, and PTEN.

55. The method of claim 40, the method further comprising measuring expression of AR, BRCA2, EGFR, ERBB2, ERBB3, FASN, and PTEN.

56. The method of claim 49, wherein the composition comprises polynucleotides specific for at least one gene selected from the group consisting of AR, BRCA2, EGFR, ERBB2, ERBB3, FASN, and PTEN.

57. The method of claim 49, wherein the composition comprises polynucleotides specific for at least two genes selected from the group consisting of AR, BRCA2, EGFR, ERBB2, ERBB3, FASN, and PTEN.

58. The method of claim 49, wherein the composition comprises polynucleotides specific for at least three genes selected from the group consisting of AR, BRCA2, EGFR, ERBB2, ERBB3, FASN, and PTEN.

59. The method of claim 49, wherein the composition comprises polynucleotides specific for at least four genes selected from the group consisting of AR, BRCA2, EGFR, ERBB2, ERBB3, FASN, and PTEN.

60. The method of claim 49, wherein the composition comprises polynucleotides specific for at least five genes selected from the group consisting of AR, BRCA2, EGFR, ERBB2, ERBB3, FASN, and PTEN.

61. The method of claim 49, wherein the composition comprises polynucleotides specific for at least six genes selected from the group consisting of AR, BRCA2, EGFR, ERBB2, ERBB3, FASN, and PTEN.

62. The method of claim 49, wherein the composition comprises polynucleotides specific for AR, BRCA2, EGFR, ERBB2, ERBB3, FASN, and PTEN.

63. The method of claim 40, wherein at least one polynucleotide, or each polynucleotide, independently comprises at least one non-naturally occurring nucleotide base.

64. The method of claim 63, wherein the non-naturally occurring nucleotide base is a LNA.

65. The method of claim 40, wherein at least one polynucleotide, or each polynucleotide, independently comprises a detectable label.

66. The method of claim 40, wherein expression is measured by detecting an amplification product using fluorescence of a nucleic acid binding dye.

67. The method of claim 40, wherein expression levels are measured using an amplification procedure.

68. The method of claim 67, wherein the amplification procedure is PCR, RT-PCR, qPCR, or Loop-mediated isothermal amplification (LAMP).

69. A composition comprising double stranded polynucleotides that are 20-100 nucleotides in length and comprise a nucleic acid molecule that can bind to the sequence of 3, 4, 5, or each selected from the group consisting of CDH1, CREB3L4, PIK3C2B, RNH43, SREBF1, and ZMYM2, wherein the double stranded polynucleotides comprise (i) a detectable label, (ii) a non-naturally occurring nucleotide, or (iii) comprise a nucleic acid binding dye bound thereto.

70. The composition of claim 69, wherein the composition further comprises a nucleic acid molecule that can bind to 1, 2, 3, 5, 6, or each selected from the group consisting of AR, BRCA2, EGFR, ERBB2, ERBB3, FASN, and PTEN.

71. The composition of claim 69, wherein the dye comprises SYBR Green, SYBR Green II, SYBR Green I, SYBR Gold, SYT09, EvaGreen, or DIAMOND™ Nucleic Acid Dye.
